(19) United States
(12) Patent Application Publication
(10) Pub. No.: US 2009/0311187 A1
(43) Pub. Date: Dec. 17, 2009

(54) Methods for predicting patient response to modulation of the Co-stimulatory pathway

(75) Inventors: David M. Berman, Princeton, NJ (US); Scott D. Chasalow, Pennington, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/455,108

(22) Filed: May 28, 2009

Related U.S. Application Data
(60) Provisional application No. 61/057,018, filed on May 29, 2008.

(51) Int. Cl. A61K 49/00; A61P 43/00

(52) U.S. Cl. 424/9.2

(57) ABSTRACT

The invention described herein relates to diagnostic and therapeutic methods and compositions useful for predicting the likelihood a patient will have favorable response to the administration of a pharmaceutically acceptable amount of an activator of the immune system (e.g, T-cells).

This application claims benefit to provisional application U.S. Ser. No. 61/057,018 filed May 29, 2008, under 35 U.S.C. 119(e). The entire teachings of the referenced applications are incorporated herein by reference.

### FIELD OF THE INVENTION

The invention described herein relates to diagnostic and therapeutic methods and compositions useful for predicting the likelihood a patient will have favorable response to the administration of a pharmaceutically acceptable amount of an activator of the immune system (e.g. T-cells).

### BACKGROUND OF THE INVENTION

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Anticancer agents have been classified based upon their mechanism of action, and are often referred to as chemotherapeutics. The combination of chemotherapeutics with immune modulating agents has been gaining increasing acceptance in the oncology field.

The vertebrate immune system requires multiple signals to achieve optimal immune activation; see, e.g., Janeway, Cold Spring Harbor Symp. Quant. Biol., 54:1-14 (1989); Paul, W. E., ed., Fundamental Immunology, 4th edition Raven Press, N.Y. (1998), particularly chapters 12 and 13, pp. 411-478. Interactions between T lymphocytes (T cells) and antigen presenting cells (APC's) are essential to the immune response. Levels of many cohesive molecules found on T cells and APC's increase during an immune response (Springer et al., Ann. Rev. Immunol., 5:223-252 (1987); Shaw et al., Curr. Opin. Immunol., Kindt and Long, eds., 1:92-97 (1988)); and Hemler, Immunology Today, 9:109-113 (1988)). Increased levels of these molecules may help explain why activated APC's are more effective at stimulating antigen-specific T cell proliferation than are resting APC's (Kaiuchi et al., J. Immunol., 131:109-114 (1983); Kreiger et al., J. Immunol., 135:2937-2945 (1985); McKenzie, J. Immunol., 141:2907-2911 (1988); and Hawrylowicz et al., J. Immunol., 141:4083-4088 (1988)).

T cell immune response is a complex process that involves cell-cell interactions (Springer et al., Ann. Rev. Immunol., 5:223-252 (1987)), particularly between T and accessory cells such as APC's, and production of soluble immune mediators (cytokines or lymphokines) (Dinarello, New Engl. J. Med., 317:940-945 (1987); Sallusto, J. Exp. Med., 179:11094118 (1997)). This response is regulated by several T-cell surface receptors, including the T-cell receptor complex (Weiss, Ann. Rev. Immunol., 4:593-619 (1986)) and other “accessory” surface molecules (Allison, Curr. Opin. Immunol., 6:414-419 (1994); Springer (1987), supra). Many of these accessory molecules are naturally occurring cell surface differentiation (CD) antigens defined by the reactivity of monoclonal antibodies on the surface of cells (McMichael, ed., Leukocyte Typing Iff, Oxford Univ. Press, Oxford, N.Y. (1987)).

Early studies suggested that B lymphocyte activation requires two signals (Bretscher, Science, 169:1042-1049 (1970)) and now it is believed that all lymphocytes require two signals for their optimal activation, an antigen specific or clonal signal, as well as a second, antigen non-specific signal. (Janeway, supra). Freeman (J. Immunol., 143:2714-2722 (1989)) isolated and sequenced a cDNA clone encoding a B cell activation antigen recognized by MAb B7 (Freeman, J. Immunol., 13 8:3260 (1987)). COS cells transfected with this cDNA have been shown to stain by both labeled MAb B7 and MAb BB-1 (Clark, Human Immunol., 16:100-113 (1986); Yokochi, J. Immunol., 128:823 (1981); Freeman et al. (1989), supra; Freeman et al. (1987), supra). In addition, expression of this antigen has been detected on cells of other lineages, such as monocytes (Freeman et al., (1989) supra).

T helper cell (Th) antigenic response requires signals provided by APC's. The first signal is initiated by interaction of the T cell receptor complex (Weiss, J. Clin. Invest., 86:1015 (1990)) with antigen presented in the context of class 1I major histocompatibility complex (MHC) molecules on the APC (Allen, Immunol. Today, 8:270 (1987)). This antigen-specific signal is not sufficient to generate a full response, and in the absence of a second signal may actually lead to clonal inactivation or anergy (Schwartz, Science, 248:1349 (1990)). The requirement for a second “costimulatory” signal provided by the MHC has been demonstrated in a number of experimental systems (Schwartz, supra; Weaver et al., Immunol. Today, 11:49 (1990)).

CD28 antigen, a homodimeric glycoprotein of the immunoglobulin superfamily (Aruffo et al., Proc. Natl. Acad. Sci., 84:8573-8577 (1987)), is an accessory molecule found on most mature human T cells (Damle et al., J. Immunol., 131:2296-2300 (1983)). Current evidence suggests that this molecule functions in an alternative T cell activation pathway distinct from that initiated by the T-cell receptor complex (June et al., Mol. Cell. Biol., 7:4472-4481 (1987)). Monoclonal antibodies (MAbs) reactive with CD28 antigen can augment T cell responses initiated by various polyclonal stimuli (reviewed by June et al., supra). These stimulatory effects may result from MAb-induced cytokine production (Thompson et al., Proc. Natl. Acad. Sci., 86:1333-1337 (1989); and Lindsten et al., Science, 244:339-343 (1989)) as a consequence of increased mRNA stabilization (Lindsten et al. (1989), supra). Anti-CD28 mAbs can also have inhibitory effects, i.e., they can block autologous mixed lymphocyte reactions (Damle et al., Proc. Natl. Acad. Sci., 78:5096-6001 (1981)) and activation of antigen-specific T cell clones (Lesslauer et al., Eur. J. Immunol., 16:1289-1296 (1986)).

Some studies have indicated that CD28 is a counter-receptor for the B cell activation antigen, B7/BB-1 (Linsley et al., Proc. Natl. Acad. Sci. USA, 87:5031-5035 (1990)). The B7/BB-I antigen is hereafter referred to as the “B7 antigen”. The B7 ligands are also members of the immunoglobulin superfamily but have, in contrast to CD28, two Ig domains in their extracellular region, an N-terminal variable (V)-like domain followed by a constant (C)-like domain.

Delivery of a non-specific costimulatory signal to the T cell requires at least two homologous B7 family members found on APC's, B7-1 (also called B7, B7.1, or CD80) and B7-2 (also called B7.2 or CD86), both of which can deliver costimulatory signals to T cells via CD28. Costimulation through CD28 promotes T cell activation.

CD28 has a single extracellular variable region (V)-like domain (Aruffo and Seed, supra). A homologous molecule, CTLA-4, has been identified by differential screening of a murine cytolytic-T cell cDNA library (Brunet, Nature, 328:267-270 (1987)).

CTLA-4 (CD152) is a T cell surface molecule that was originally identified by differential screening of a murine cytolytic T cell cDNA library (Brunet et al., Nature, 328:267-270(1987)). CTLA-4 is also a member of the immunoglobulin (Ig) superfamily; CTLA-4 comprises a single extracellular Ig domain. Researchers have reported the cloning and mapping of a gene for the human counterpart of CTLA-4 (Dariavach et al., Eur. J. Immunol., 18:1901-1905 (1988)) to the same chromosomal region (2q33-34) as CD28 (Lafage-Pochitaloff et al., Immunogenetics, 31:198-201 (1990)). Sequence comparison between this human CTLA-4 DNA and that encoding CD28 proteins reveals significant homology of sequence, with the greatest degree of homology in the juxtamembrane and cytoplasmic regions (Brunet et al. (1988), supra; Dariavach et al. (1988), supra).

The CTLA-4 is inducibly expressed by T cells. It binds to the B7-family of molecules (primarily CD80 and CD86) on antigen-presenting cells (Chambers et al., Ann. Rev Immunol., 19:565-594 (2001)). When triggered, it inhibits T-cell proliferation and function. Mice genetically deficient in CTLA-4 develop lymphoproliferative disease and autoimmunity (Tivol et al., Immunity., 3:541-547 (1995)). In pre-clinical models, CTLA-4 blockade also augments anti-tumor immunity (Leach et al., Science, 271:1734-1736 (1996); van Elsas et al., J. Exp. Med., 190:355-366 (1999)). These findings led to the development of antibodies that block CTLA-4 for use in cancer immunotherapy.

Blockade of CTLA-4 by a monoclonal antibody leads to the expansion of all T cell populations, with activated CD4⁺ and CD8⁺ T cells mediating tumor cell destruction (Melero et al., Nat Rev Cancer 2007;7:95-106; Wolchok et al., The Oncologist 2008;13 (suppl. 4):2-9). The antitumor response that results from the administration of anti-CTLA-4 antibodies is believed to be due to an increase in the ratio of effector T cells to regulatory T cells within the tumor microenvironment, rather than simply from changes in T cell populations in the peripheral blood (Quezada et al., J Clin Invest 2006;116:1935-45). One such agent under clinical investigation is ipilimumab.

Ipilimumab (previously MDX-010; Medarex Inc.) is a fully human anti-human CTLA-4 monoclonal antibody that blocks the binding of CTLA-4 to CD80 and CD86 expressed on antigen presenting cells, thereby, blocking the negative down-regulation of the immune responses elicited by the interaction of these molecules. Initial studies in patients with melanoma showed that ipilimumab could cause objective durable tumor regressions (Phan et al., Proc. Natl. Acad. Sci. USA, 100:8372-8377 (2003)). Also, reductions of serum tumor markers were seen for some patients with ovarian or prostate cancer (Hodi et al., Proc. Natl. Acad. Sci. USA, 100:4712-4717 (2003)). More recently, ipilimumab has demonstrated antitumor activity in patients with advanced melanoma (Weber et al., J Clin Oncol 2008;26:5950-56; Weber, Cancer Immunol. Immunother 2009;58:823-30). However, a marker of early immune activation with ipilimumab has yet to be identified. Accordingly, there is a need in the art to identify patients who may have a favorable response to anti-CTLA-4 therapy.

One potential candidate is absolute lymphocyte count (ALC). ALC is a standard, clinically accepted blood cell parameter that is routinely measured by physicians prior to therapeutic treatment for certain leukemias and lymphomas. Recently, ALC has been associated with clinical pathology for several types of leukemias and lymphomas. Specifically, Porrata et al. have shown that recovery of ALC post auto-transplant in lymphoma and myeloma patients is predictive of relapse (Blood, 98:579-585 (2001)). In addition, there is also some evidence that ALC at diagnosis and prior to anti CD-20 targeted therapy may be a useful prognostic marker in follicular lymphoma (Siddiqui et al., Br. J. Haematology, 134:596-601 (2006); Behl et al., Br. J. Haematology, 137:409-415 (2007)). However, the predictive value of the absolute lymphocytic count (ALC) has been a recent matter of debate in non-Hodgkin-lymphoma (Leukemia, 21:2227-2230 (2007)).

Nonetheless, the predictive value of ALC for leukemias has been gaining acceptance. For example, De Angulo et al. show that ALC is a significant independent predictor of relapse and survival in acute myeloblastic leukemia (AML) and acute lymphoblastic leukemia (ALL) (Cancer, 112(2):407-415 (2008)), which was also observed by Behl et al., (Leukemia, 20(1):29-34 (2006)).

More recently, low ALC at diagnosis and/or at specific times following treatment has been found to be a negative factor for survival in a number of hematological malignancies and solid tumors, including diffuse-large-B-cell-lymphoma (Cox et al., Leuk Lymphoma 2008;49: 1745-51), high-risk Ewing sarcoma (De Angulo et al., J Pediatr Hematol Oncol 2007;29:48-52), acute lymphoblastic leukemia and acute myeloblastic leukemia (De Angulo et al., Cancer 2008;112:407-15), multiple myeloma (Ege et al., Br J Haematol 2008;141:792-98), and brain metastases from breast cancer (Claude et al., Radiother Oncol 2005;76:334-39).

However, use of ALC has been limited to predicting patient survival, but has not been previously shown to be an indicator for predicting patient response to specific therapies, let alone specific immunomodulatory therapies.

The present inventors have discovered, for the first time, that change in absolute lymphocyte count over time in patients receiving anti-CTLA-4 therapy for non-blood cancers, such as melanoma, is useful for predicting the likelihood a patient will achieve a favorable response to immunotherapy.

### SUMMARY OF THE INVENTION

The present invention provides a method for predicting the likelihood a patient will have a favorable response to therapy that activates T-cells for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a negative slope have a lower likelihood of achieving a favorable response to said therapy. Patients who achieved a favorable response had a positive slope, and, on average, a higher positive slope than patients who did not achieve a favorable response. Accordingly, patients with a negative slope may require a more aggressive dosing regimen of a therapeutically acceptable amount of said therapy, either alone or in combination with other agents to achieve a favorable response.

The present invention provides a method for predicting the likelihood a patient will have a favorable response to therapy involving the inhibition of CTLA-4 for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a negative slope have a lower likelihood of achieving a favorable response to said therapy. Patients who achieved a favorable response had a positive slope, and, on average, a higher positive slope than patients who did not achieve a favorable response. Accordingly, patients with a negative slope may require a more aggressive dosing regimen of a therapeutically acceptable amount of said therapy, either alone or in combination with other agents to achieve a favorable response. Accordingly, patients with a negative slope may require a more aggressive dosing regimen of a therapeutically acceptable amount of said therapy, either alone or in combination with other agents to treat said disorder.

The present invention provides a method for predicting the likelihood a patient will have a favorable response to therapy that activates T-cells for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a negative slope have a lower likelihood of achieving a favorable response to said therapy. Patients who achieved a favorable response had a positive slope, and, on average, a higher positive slope than patients who did not achieve a favorable response. Accordingly, patients with a negative slope may require a more aggressive dosing regimen of a therapeutically acceptable amount of said therapy, either alone or in combination with other agents to achieve a favorable response. Accordingly, patients with a negative slope may require a more aggressive dosing regimen of a therapeutically acceptable amount of said therapy, either alone or in combination with other agents to treat said disorder.

The present invention provides a method for predicting the likelihood a patient will have a favorable response to therapy involving the administration of an anti-CTLA-4 antibody for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a negative slope have a lower likelihood of achieving a favorable response to said therapy. Patients who achieved a favorable response had a positive slope, and, on average, a higher positive slope than patients who did not achieve a favorable response. Accordingly, patients with a negative slope may require a more aggressive dosing regimen of a therapeutically acceptable amount of said therapy, either alone or in combination with other agents to achieve a favorable response. Accordingly, patients with a negative slope may require a more aggressive dosing regimen of a therapeutically acceptable amount of said therapy, either alone or in combination with other agents to treat said disorder.

The present invention provides a method for predicting the likelihood a patient will have a favorable response to therapy involving the administration of ipilimumab for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a negative slope have a lower likelihood of achieving a favorable response to said therapy. Patients who achieved a favorable response had a positive slope, and, on average, a higher positive slope than patients who did not achieve a favorable response. Accordingly, patients with a negative slope may require a more aggressive dosing regimen of a therapeutically acceptable amount of said therapy, either alone or in combination with other agents to achieve a favorable response. Accordingly, patients with a negative slope may require a more aggressive dosing regimen of a therapeutically acceptable amount of said therapy, either alone or in combination with other agents to treat said disorder.

The present invention provides a method for predicting the likelihood a patient will have a favorable response to therapy involving the modulation of the co-stimulatory pathway for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a negative slope have a lower likelihood of achieving a favorable response to said therapy. Patients who achieved a favorable response had a positive slope, and, on average, a higher positive slope than patients who did not achieve a favorable response. Accordingly, patients with a negative slope may require a more aggressive dosing regimen of a therapeutically acceptable amount of said therapy, either alone or in combination with other agents to achieve a favorable response. Accordingly, patients with a negative slope may require a more aggressive dosing regimen of a therapeutically acceptable amount of said therapy, either alone or in combination with other agents to treat said disorder, wherein said disorder is melanoma.

The present invention provides a method for predicting the likelihood a patient will have a favorable response to therapy that activates T-cells for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder.

The present invention provides a method for predicting the likelihood a patient will have a favorable response to therapy involving the inhibition of CTLA-4 for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder.

The present invention provides a method for predicting the likelihood a patient will have a favorable response to therapy involving the administration of an anti-CTLA-4 antibody for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder.

The present invention provides a method for predicting the likelihood a patient will have a favorable response to therapy involving the administration of ipilimumab for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder.

The present invention provides a method for predicting the likelihood a patient will have a favorable response to therapy involving the modulation of the co-stimulatory pathway for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder, wherein said disorder is melanoma.

The present invention provides a method for predicting the likelihood a patient will have a favorable response to therapy involving the inhibition of CTLA-4 for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder, wherein said disorder is melanoma.

The present invention provides a method for predicting the likelihood a patient will have a favorable response to therapy involving the administration of an anti-CTLA-4 antibody for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder, wherein said disorder is melanoma.

The present invention provides a method for predicting the likelihood a patient will have a favorable response to therapy involving the administration of ipilimumab for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder, wherein said disorder is melanoma.

The present invention provides a method for predicting the likelihood a patient will have a favorable response to therapy involving the inhibition of CTLA-4 for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder, wherein said disorder is melanoma, and wherein said other agent is selected from the group consisting of: chemotherapy, a tubulin stabilizing agent; pacitaxel; an epothilone; a taxane; Dacarbazine; PARAPLATIN®; Docetaxel; one or more peptide vaccines; MDX-1379 Melanoma Peptide Vaccine; one or more gp100 peptide vaccine; fowlpox-PSA-TRICOM™ vaccine; vaccinia-PSA-TRICOM™ vaccine; MART-1 antigen; sargramostim; ticilimumab; and/or Combination Androgen Ablative Therapy.

The present invention provides a method for predicting the likelihood a patient will have a favorable response to therapy involving the inhibition of CTLA-4 for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder, wherein said disorder is melanoma, and wherein said more aggressive dosing regimen involves the administration of 10, 20, 30, 40, 50, 60, 70, 80, 90, or 95% more than the prescribed dose of said therapy, or 1.5×, 2×, 2.5×, 3×, 3.5×, 4×, 4.5×, or 5× more than the prescribed dose of said therapy, and alternatively wherein said increased dosing frequency is in combination with another agent.

The present invention provides a method for treating a patient with therapy involving the modulation of the co-stimulatory pathway for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope may be administered said therapy alone at the recommended dose, whereas patients that have a negative slope may require a more aggressive dosing regimen of a therapeutically acceptable amount of said therapy, either alone or in combination with other agents to treat said disorder.

The present invention provides a method for treating a patient with therapy involving the inhibition of the CTLA-4 for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder.

The present invention provides a method for treating a patient with therapy administration of an anti-CTLA-4 antibody for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder.

The present invention provides a method for treating a patient with a therapy comprising the administration of ipilimumab for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy. Patients who achieved a favorable response had, on average, a higher slope than patients who did not achieve a favorable response. Accordingly, patients with a negative slope may require a more aggressive dosing regimen of a therapeutically acceptable amount of said therapy, either alone or in combination with other agents to treat said disorder, wherein said disorder is melanoma.

The present invention provides a method for treating a patient with a therapy comprising the administration of a chemotherapy regimen for a disorder, including cancer, comprising the steps of: (i) measuring absolute lymphocyte count of patient samples collected over time prior to, about the same time as, and/or subsequent to administration of said therapy; and (ii) calculating a slope of said absolute lymphocyte count, wherein patients that have a positive slope have a higher likelihood of achieving a favorable response to said therapy, whereas patients that have a negative slope have a lower likelihood of achieving a favorable response to said therapy, said patients may require a more aggressive dosing regimen of a therapeutically acceptable amount of said therapy, either alone or in combination with other agents to treat said disorder, wherein said disorder is melanoma and/or lung cancer.

The present invention also is directed to a kit for use in determining a treatment strategy for an individual with a disorder, including cancer, comprising a means for measuring absolute lymphocyte counts over time, and calculating a slope for said absolute lymphocyte counts; and optionally instructions for use and interpretation of the kit results, wherein said treatment strategy comprises administration of a therapeutically effective amount of a co-stimulatory pathway modulator, or a pharmaceutically acceptable salt, hydrate or solvate thereof.

The present invention also is directed to a kit for use in determining a treatment strategy for an individual with a disorder, including cancer, comprising a means for measuring absolute lymphocyte counts over time, and calculating a slope for said absolute lymphocyte counts; and optionally instructions for use and interpretation of the kit results, wherein said treatment strategy comprises administration of a therapeutically effective amount of a CTLA-4 inhibitor, or a pharmaceutically acceptable salt, hydrate or solvate thereof.

The present invention also is directed to a kit for use in determining a treatment strategy for an individual with a disorder, including cancer, comprising a means for measuring absolute lymphocyte counts over time, and calculating a slope for said absolute lymphocyte counts; and optionally instructions for use and interpretation of the kit results, wherein said treatment strategy comprises administration of a therapeutically effective amount of an anti-CTLA-4 antibody, or a pharmaceutically acceptable salt, hydrate or solvate thereof.

The present invention also is directed to a kit for use in determining a treatment strategy for an individual with a disorder, including cancer, comprising a means for measuring absolute lymphocyte counts over time, and calculating a slope for said absolute lymphocyte counts; and optionally instructions for use and interpretation of the kit results, wherein said treatment strategy comprises administration of a therapeutically effective amount of an ipilimumab, or a pharmaceutically acceptable salt, hydrate or solvate thereof.

### BRIEF DESCRIPTION OF THE FIGURES/DRAWINGS

FIG. 1. Fitted Mean ALC Versus Weeks Since First Dose. Fitted mean ALC versus weeks since first dose, by dose, is shown. Thick curves show fitted means. Thin curves are bounds of 95% confidence bands for the mean. Nominal dosing dates were at 0, 3, 6, and 9 weeks (dashed vertical lines). All patients in studies CA184-007, -008, and -022 were included, except the 2 patients as noted in Example 1 (n=482 patients, 2715 data points total). All time points between 4 weeks prior to and 12 weeks after first dose were included. An extended linear model was fit by REML, with spatial exponential within-patient correlation structure (Euclidean distance), and within-patient variances inversely proportional to the number of ALC measures on a given day. The change in ALC over time was modeled using splines with a knot at 0: linear before 0 and cubic after. As shown, the mean ALC slope for patients administered 10 mg/kg of ipilimumab was greater than, and statistically significantly different from, that for patients who were administered 3.0 mg/kg or 0.3 mg/kg.

FIG. 2. Estimated Change in ALC Per Week (slope) Versus Estimated ALC at Date of First Dose for CA184-007, -008, and -022. Estimated change in ALC per week (slope) versus estimated ALC at date of first dose (intercept), by dose, for studies CA184-007, -008, and -022, is shown. Each point is one patient. For each patient, slope and intercept were estimated by simple linear regression. Solid horizontal lines in each panel give 25th, 50th, and 75th percentiles of the slopes in the panel. Includes all patients with known date of first dose, at least 1 post-first-dose ALC value, and at least 2 ALC values between study days −28 and 84 (weeks −4 and 12), inclusive (n=462). Only ALC values between study days −28 and 84, inclusive, were included in the analyses. As shown, the mean change in ALC per week (slope) for patients administered 10 mg/kg of ipilimumab was greater than, and statistically significantly different from, that for patients who were administered 3.0 mg/kg or 0.3 mg/kg.

FIG. 3. Estimated Change in ALC Per Week (slope) Versus Estimated ALC at Date of First Dose for study CA184-022 only. Estimated change in ALC per week (slope) versus estimated ALC at date of first dose (intercept), by dose, for study CA184-022 only, is shown. Each point is one patient. For each patient, slope and intercept were estimated by simple linear regression. Solid horizontal lines in each panel give 25th, 50th, and 75th percentiles of the slopes in the panel. Includes all patients with known date of first dose, at least 1 post-first-dose ALC value, and at least 2 ALC values between study days −28 and 84 (weeks −4 and 12), inclusive (n=201). Only ALC values between study days −28 and 84, inclusive, were included in the analyses. Even when restricted to this single study, an association between ALC slope and dose was apparent.

FIG. 4. Estimated Change in ALC Per Week (slope) Versus Estimated ALC at Date of First Dose by Dose and Response Category for CA184-007, -008, and -022. Estimated change in ALC per week (slope) versus estimated ALC at date of first dose (intercept), by dose and Response Category, for studies CA184-007, -008, and -022, is shown. Each point is one patient. For each patient, slope and intercept were estimated by simple linear regression. Solid horizontal lines in each panel give 25th, 50th, and 75th percentiles of the slopes in the panel. Includes all response-evaluable patients with known date of first dose, at least 1 post-first-dose ALC value, and at least 2 ALC values between study days −28 and 84 (weeks −4 and 12), inclusive (n=379). Only ALC values between study days −28 and 84, inclusive, were included in the analyses. As shown, the difference in mean slope between the Benefit and Non-Benefit groups for patients who received 10 mg/kg ipilimumab was highly, statistically significant.

FIG. 5. Estimated Change in ALC Per Week (slope) Versus Estimated ALC at Date of First Dose by Dose and irResponse Category for CA184-007, -008, and -022. Estimated change in ALC per week (slope) versus estimated ALC at date of first dose (intercept), by dose and irResponse Category, for studies CA184-007, -008, and -022, is shown. Each point is one patient. For each patient, slope and intercept were estimated by simple linear regression. Solid horizontal lines in each panel give 25th, 50th, and 75th percentiles of the slopes in the panel. Includes all response-evaluable patients with known date of first dose, at least 1 post-first-dose ALC value, and at least 2 ALC values between study days −28 and 84 (weeks −4 and 12), inclusive (n=379). Only ALC values between study days −28 and 84, inclusive, were included in the analyses As shown, the difference in mean slope between the irResponse categories for patients who received 10 mg/kg ipilimumab was highly, statistically significant.

FIG. 6. Estimated Change in ALC Per Week (slope) Versus Estimated ALC at Date of First Dose, by Dose and Response Category, for Study CA184-004 Only. Estimated change in ALC per week (slope) versus estimated ALC at date of first dose (intercept), by dose and response category, for study CA184-004 only, is shown. Each point is one patient. For each patient, slope and intercept were estimated by simple linear regression. Solid horizontal lines in each panel give 25th, 50th, and 75th percentiles of the slopes in the panel. Includes all patients with known date of first dose, at least one post-first-dose ALC value, and at least two ALC values between study days −28 and 84 (weeks −4 and 12), inclusive (n=65). Only ALC values between study days −28 and 84, inclusive, were included in the analyses. Positive associations between ALC slope and dose, and ALC slope and response category, were seen in this study, similar to those seen in the combined analysis of studies CA184-007, CA 184-008 and CA 184-022.

FIG. 7. Relationship between antitumor response and ipilimumab steady-state trough concentrations (Cminₛₛ). In both (A) and (B), the solid line and shaded area represent median values of model prediction and 90% bootstrap CI (n=500). Horizontal box plots represent the distribution of Cminₛₛ at each dose group: boxes (25th, 50th, and 75th percentile) and whiskers (5th and 95th percentiles). (A) Model predicted probability with 90% CI of BOR (CR or PR) vs. Cminₛₛ. The probability of BOR increased from 4.9% to 19.5% at the 5th and 95th percentiles of Cminₛₛ. The results from a predictive check indicated a good agreement between the model-predicted probability of BOR responders and the observed proportion of BOR responders (data not shown). (B) Model predicted probability with 90% CI of irCA vs. Cminₛₛ. The probabilities of achieving irCA were more than double that of BOR at 25th and 75th percentiles of Cminₛₛ.

### DETAILED DESCRIPTION OF THE INVENTION

The present invention is based, in part, on data from four phase II clinical trials that demonstrated patients who exhibited a positive slope, for measurements involving absolute lymphocyte counts (“ALC” herein) as a function of time after the administration of the anti-CTLA-4 antibody, ipilimumab, had a higher likelihood of achieving a clinical benefit and/or immune-related response. Generally, patients who exhibited a negative slope for the absolute lymphocyte count as a function of time after the administration of ipilimumab, failed to achieve a clinical benefit. However, one of the 91 patients who exhibited a negative slope did achieve a clinical benefit.

Accordingly, the slope of ALC is positively associated with, and is thus useful as a predictive indicator for, clinical benefit and/or immune-related response for patients receiving a co-stimulatory pathway modulator, such as for example, ipilimumab. In addition, the slope of ALC also is positively associated with, and is thus useful as a predictive indicator for, clinical benefit and/or immune-related response for patients receiving an immunostimulant and/or T-cell activator, such as for example, ipilimumab.

For the purposes of the present invention, the phrase “positively associated” refers to a general condition where a higher ALC slope value for a given patient suggests that the patient will have a correspondingly higher likelihood of achieving a clinical benefit, relative to a patient who has a lower ALC slope value.

In addition, a negative slope of ALC is useful as a predictive indicator for identifying patients who may have a lower likelihood of responding to or achieving clinical benefit and/or immune-related response to the administration of a co-stimulatory pathway modulator, such as for example, ipilimumab. In addition, a negative slope of ALC may be useful for identifying patients who may require more aggressive dosing regimens of a co-stimulatory pathway modulator, or combination therewith, in order to achieve clinical benefit and/or immune-related response to co-stimulatory pathway modulator therapy.

Measurement of the slope of ALC, both positive and negative, may also be useful as a predictive indicator for identifying patients who may respond to other types of therapies beyond merely co-stimulatory pathway modulators, which include, for example, but are not limited to, chemotherapy.

The use of ALC slope as a diagnostic is also useful for, among other things, assisting health care professionals in developing tailored treatment regimens suitable for the condition(s) presented herein, particularly for the treatment of melanoma.

The teachings of the present invention are believed to be the first association between the slope of ALC and patient response to a specific therapy, in general, and specifically response to a co-stimulatory pathway modulator, such as ipilimumab. While the use of ALC (but not slope), as an indicator for predicting overall survival for a select, and limited number of cancers, including certain hematological malignancies, ALL, AML, high-risk Ewing sarcoma, multiple myeloma, and brain metastases from breast cancer, is known, it has not been used as a predictive indicator for predicting patient response(s) to therapeutic intervention of such disorders—rather, it has only been used to predict survival. In addition, the use of ALC slope as a predictive indicator of patient response to an immunomodulatory agent has also not been previously described.

The use of ALC as a indicator for predicting overall survival for these cancers appears to have been limited to measuring the base-line ALC prior to treatment, and did not involve measuring ALC as a function of time (e.g., slope) during the period of therapeutic intervention, let alone applying the value of the slope to make a prediction of the likelihood a patient will achieve a clinical benefit based upon whether the slope is positive or negative, as is described herein. The use of ALC, but not slope, subsequent to therapeutic administration has been used to predict patient survival (see DeAngelo et al., J. Pediatr. Hemat. Oncol., 29(1):48-52 (2007); DeAngelo et al., Cancer, 112(2):407-415 (2007), and Behl et al., Br. J. Haematology, 137:409-415 (2007)), but such applications of ALC have relied upon the value of ALC at the time of measurement as a threshold (i.e., whether ALC was above or below a certain numerical limit)—the change of ALC over time (e.g., slope) has not been described heretofore. The present invention is directed to the use of ALC slope as a predictive indicator of patient response to immunomodulatory therapy.

For the purposes of the present invention, the value of a patient's ALC may be measured beginning on or about the day of first therapeutic dose, and continue at a regular frequency for a period of time, as outlined herein or otherwise as requested by a health care professional. A patient's ALC may optionally be measured prior to the first therapeutic dose as well. In one embodiment of the present invention, the value of a patient's ALC may be measured monthly, bi-weekly, weekly, intra-weekly, or even as frequently as daily (herein referred to as “ALC measurement frequency”). After a given interval of time (herein referred to as “ALC slope interval”), the slope may then be calculated using two or more time points residing within the ALC slope interval for use in making a predictive prediction regarding an individual patient's therapeutic response.

The length of the ALC slope interval may depend, in part, on the ALC measurement frequency, with shorter frequencies permitting shorter intervals, in general. In one embodiment of the present invention, the ALC slope interval may be about 24 weeks. In another embodiment of the present invention, the ALC slope interval may be about 20 weeks. In another embodiment of the present invention, the ALC slope interval may be about 18 weeks. In another embodiment of the present invention, the ALC slope interval may be about 15 weeks. In another embodiment of the present invention, the ALC slope interval may be about 12 weeks. In another embodiment of the present invention, the ALC slope interval may be about 11 weeks. In another embodiment of the present invention, the ALC slope interval may be about 10 weeks. In another embodiment of the present invention, the ALC slope interval may be about 9 weeks. In another embodiment of the present invention, the ALC slope interval may be about 8 weeks. In another embodiment of the present invention, the ALC slope interval may be about 7 weeks. In another embodiment of the present invention, the ALC slope interval may be about 6 weeks. In another embodiment of the present invention, the ALC slope interval may be about 5 weeks. In another embodiment of the present invention, the ALC slope interval may be about 4 weeks. In another embodiment of the present invention, the ALC slope interval may be about 3 weeks. In another embodiment of the present invention, the ALC slope interval may be about 2 weeks. In another embodiment of the present invention, the ALC slope interval may be about 1 week. In this context, the term “about” shall be construed to mean±1, 2, 3, 4, 5, 6, or 7 days more or less than the stated ALC slope interval.

In one embodiment, the assignment of the slope to being either positive or negative may be made after the ALC slope for the ALC slope interval of interest has been calculated based upon whether the value of the slope is above or below a threshold rate of change (referred to herein as “ALC slope threshold”). For the purposes of the present invention, the ALC slope threshold for assignment of the slope to be positive is zero. For example, if a slope for a given patient within a given ALC slope interval is zero, or if it is greater than zero, then that patient will be assigned as having a positive slope. Likewise, if a slope for a given patient within a given ALC slope interval is less than zero, then that patient will be assigned as having a negative slope. In one embodiment of the present invention, the ALC slope threshold may be about 0. In another embodiment of the present invention, the ALC slope threshold may be about 0.001. In another embodiment of the present invention, the ALC slope threshold may be about 0.005. In another embodiment of the present invention, the ALC slope threshold may be about 0.01. In another embodiment of the present invention, the ALC slope threshold may be about 0.015. In another embodiment of the present invention, the ALC slope threshold may be about 0.020. In another embodiment of the present invention, the ALC slope threshold may be about 0.025. In another embodiment of the present invention, the ALC slope threshold may be about 0.030. In another embodiment of the present invention, the ALC slope threshold may be about 0.035. In another embodiment of the present invention, the ALC slope threshold may be about 0.040. In another embodiment of the present invention, the ALC slope threshold may be about 0.045. In another embodiment of the present invention, the ALC slope threshold may be about 0.050. In another embodiment of the present invention, the ALC slope threshold may be about 0.055. In another embodiment of the present invention, the ALC slope threshold may be about 0.060. In another embodiment of the present invention, the ALC slope threshold may be about 0.065. In another embodiment of the present invention, the ALC slope threshold may be about 0.070. In another embodiment of the present invention, the ALC slope threshold may be about 0.075. In another embodiment of the present invention, the ALC slope threshold may be about 0.080. In another embodiment of the present invention, the ALC slope threshold may be about 0.085. In another embodiment of the present invention, the ALC slope threshold may be about 0.090. In another embodiment of the present invention, the ALC slope threshold may be about 0.095. In another embodiment of the present invention, the ALC slope threshold may be about 0.10. In another embodiment of the present invention, the ALC slope threshold may be about 0.15. In another embodiment of the present invention, the ALC slope threshold may be about 0.2. In this context, the term “about” should be construed to mean ±0.001, ±0.002, ±0.003, ±0.004, ±0.005, ±0.006, ±0.007, ±0.008, ±0.009, ±0.01, ±0.015, ±0.02, ±0.025, or ±0.03 of the stated ALC slope threshold value.

In another embodiment, an estimate of the likelihood of clinical benefit may be based on ALC slope as a continuous measure, without reference to an ALC slope threshold, but rather using the magnitude of positive or negative value of the slope. For example, a patient having a higher ALC slope value, on average, may have a correspondingly higher likelihood of achieving a clinical benefit, relative to the patient who has a lower ALC slope value. Accordingly, a patient who has an ALC slope value of about 2.0 has a higher likelihood of achieving clinical benefit than a patient having an ALC slope of about 1.80; has a higher likelihood of achieving clinical benefit than a patient having an ALC slope of about 1.60; has a higher likelihood of achieving clinical benefit than a patient having an ALC slope of about 1.40; has a higher likelihood of achieving clinical benefit than a patient having an ALC slope of about 1.20; has a higher likelihood of achieving clinical benefit than a patient having an ALC slope of about 1.0; has a higher likelihood of achieving clinical benefit than a patient having an ALC slope of about 0.80; has a higher likelihood of achieving clinical benefit than a patient having an ALC slope of about 0.60; has a higher likelihood of achieving clinical benefit than a patient having an ALC slope of about 0.40; has a higher likelihood of achieving clinical benefit than a patient having an ALC slope of about 0.20; has a higher likelihood of achieving clinical benefit than a patient having an ALC slope of about 0.0; has a higher likelihood of achieving clinical benefit than a patient having an ALC slope of about −0.02; has a higher likelihood of achieving clinical benefit than a patient having an ALC slope of about −0.04; has a higher likelihood of achieving clinical benefit than a patient having an ALC slope of about −0.06; has a higher likelihood of achieving clinical benefit than a patient having an ALC slope of about −0.08; has a higher likelihood of achieving clinical benefit than a patient having an ALC slope of about −0.1; has a higher likelihood of achieving clinical benefit than a patient having an ALC slope of about −0.2; has a higher likelihood of achieving clinical benefit than a patient having an ALC slope of about −0.4; has a higher likelihood of achieving clinical benefit than a patient having an ALC slope of about −0.6; has a higher likelihood of achieving clinical benefit than a patient having an ALC slope of about −0.8; has a higher likelihood of achieving clinical benefit than a patient having an ALC slope of about −1.00. In this context, the term “about” should be construed to mean±0.01, ±0.02, ±0.03, ±0.04, ±0.05, ±0.06, ±0.07, ±0.08, ±0.09, ±0.1, ±0.15, ±0.2, ±0.25, ±0.3, ±0.35, ±0.4, ±0.45, or ±0.5, of the stated ALC slope value.

The present invention contemplates that any given patient response to a therapy is complex, and likely depends upon a number of factors, including, but not limited to a patient's genetic background, diet, lifestyle, or may even depend upon the presence or absence of confounding patient conditions such as the presence of other disorders at the time the therapy is administered, or that may arise during the course of therapeutic administration, etc. Such factors may obscure or delay the presentation of a true, positive ALC slope, such that the presence of such factors may cause the value of the slope to be 0 or even to be slightly negative within the ALC slope interval, which would be otherwise positive in the absence of such factors. Accordingly, for the purposes of the present invention, the definition of a positive ALC slope may also include slopes that are either at 0 or about 0, or slopes that are negative but within about ±10%, about ±5%, or even about ±1% of being about 0.

Furthermore, in certain circumstances where the ALC measurement frequency is very low, or during times when a health care professional recognizes that confounding patient factors or circumstances have affected the value of the ALC such that it may be undesirable to use one or more of the ALC values residing within the ALC slope interval, a limited number of ALC values may be used for calculating the ALC slope during the applicable ALC slope interval.

The transformation of a patient's ALC slope into a probability for predicting patient response may depend upon a number of factors, including, but not limited to the patient's health, the condition for which the patient is being treated, the therapy the patient has been administered, the dose of the therapy administered, the frequency of the dosing regiment, or any other considerations a health care professional may take into account. Nonetheless, a greater ALC slope value may be transformed into a probability that predicts a patient will have an increased probability of achieving a clinical benefit; while a lesser ALC slope value may be transformed into a probability that predicts a patient will have a decreased probability of achieving clinical benefit.

The present invention contemplates that the present invention may be carried out in a number of different modes. For example, in one mode, the present invention contemplates at least one or more of the steps of the diagnostic method being performed by a computer. For example, the calculation of a patient's ALC slope, optionally within the ALC slope interval, may be performed by a computer. In addition, the determination of whether the ALC slope is positive or negative, and optionally whether it is above or below the ALC slope threshold, may be performed by a computer. In addition, the transformation of the ALC slope, optionally in conjunction with the ALC slope threshold, into the probability of a patient achieving a clinical benefit to a therapy may be performed by a computer. One of skill in the computer programming arts could readily draft software that performs the steps of the invention algorithmically.

The computer for carrying out one mode of the present invention may comprise a CPU, ROM, standard I/O for receiving and outputting instructions and responses, algorithms for carrying out specific steps of the present invention, operating system software, and the like. The computer also may include a display means for conveying I/O information to the user (e.g., monitor, LCD, CRT, etc.), and may also include an entry means (e.g., keyboard, mouse, trackball, touch pad, etc.) to permit user interaction.

The phrase “clinical benefit” or “benefit” refers to a condition where a patient achieves a complete response; partial response; stable disease; or as otherwise described herein.

The phrase “absolute lymphocyte count” refers to the number of lymphocytes in a patient sample, calculated from the percentage of lymphocytes out of the total number of white blood cells in a patient sample multiplied by the total number of white blood cells to arrive at the “absolute” lymphocyte count. The absolute number and/or percentage of lymphocytes in any given sample may be determined using a hemocytometer, flow cytometry, or other methods known in the art.

The phrase “positive slope” or “positive ALC slope” refers to the ratio of the number of units a line rises or falls vertically (Y-axis) relative to the number of units the line moves horizontally (X-axis) from left to right that results in either a value of zero or a positive value (a value greater than 0), where the Y-axis value refers to the absolute lymphocyte count of a patient sample, and the X-axis value refers to a point in time. Calculation of the slope requires ALC measurements for at least two time points. The points may include ALC values prior to, during, and/or subsequent to the administration of a co-stimulatory pathway modulator, though preferably will include points beginning on or about the first administration and continuing for an interval of time subsequent to the administration.

The phrase “negative slope” or “negative ALC slope” refers to the ratio of the number of units a line rises or falls vertically relative to the number of units the line moves horizontally from left to right that results in a negative value (a value less than zero), where the Y-axis value refers to the absolute lymphocyte count of a patient sample, and the X-axis value refers to a point in time. The points may include ALC values prior to, during, and/or subsequent to the administration of a co-stimulatory pathway modulator, though preferably will include points beginning on or about the first administration and continuing for an interval of time subsequent to the administration.

Generally, one skilled in the art will appreciate how to calculate the slope of any given line using methods well known in the art. In its most simplistic form, a two-point, ALC slope may be calculated according to the following formula:

$$m = \frac{{y}_{1} - {y}_{2}}{{x}_{1} - {x}_{2}}$$

where y₁ represents the Y-axis value of a first point along a Cartesian coordinate, y₂ represents the Y-axis value of a second point along a Cartesian coordinate, x₁ represents the X-axis value of a first point along a Cartesian coordinate, x₂ represents the X-axis value of a second point along a Cartesian coordinate. The calculation of a slope for any given line containing more than two individual points is well within the knowledge of one skilled in the art of mathematics and basic science.

The phrase “co-stimulatory pathway modulator”, generally refers to an immunostimulant or T-cell activator, and also encompasses any agent that is capable of disrupting the ability of CD28 antigen to bind to its cognate ligand, to inhibit the ability of CTLA-4 to bind to its cognate ligand, to augment T cell responses via the co-stimulatory pathway, to disrupt the ability of B7 to bind to CD28 and/or CTLA-4, to disrupt the ability of B7 to activate the co-stimulatory pathway, to disrupt the ability of CD80 to bind to CD28 and/or CTLA-4, to disrupt the ability of CD80 to activate the co-stimulatory pathway, to disrupt the ability of CD86 to bind to CD28 and/or CTLA-4, to disrupt the ability of CD86 to activate the co-stimulatory pathway, and to disrupt the co-stimulatory pathway, in general from being activated. This necessarily includes small molecule inhibitors of CD28, CD80, CD86, CTLA-4, among other members of the co-stimulatory pathway; antibodies directed to CD28, CD80, CD86, CTLA-4, among other members of the co-stimulatory pathway; antisense molecules directed against CD28, CD80, CD86, CTLA-4, among other members of the co-stimulatory pathway; adnectins directed against CD28, CD80, CD86, CTLA-4, among other members of the co-stimulatory pathway, RNAi inhibitors (both single and double stranded) of CD28, CD80, CD86, CTLA-4, among other members of the co-stimulatory pathway, among other anti-CTLA-4 antagonists.

Suitable anti-CTLA-4 antagonist agents for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, modulators of the co-stimulatory pathway, the antibodies disclosed in PCT Publication No. WO2001/014424, the antibodies disclosed in PCT Publication No. WO2004/035607, the antibodies disclosed in U.S. Published Application No. US2005/0201994, and the antibodies disclosed in granted European Patent No. EP1212422B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication No. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145):abstract no. 2505 (2004) (antibody CP-675206); Mokyr et al., Cancer Res., 58:5301-5304 (1998), U.S. Pat. No. 5,977,318, U.S. Pat. No. 6,682,736, U.S. Pat. No. 7,109,003, and U.S. Pat. No. 7,132,281. Each of these references is specifically incorporated herein by reference for purposes of description of CTLA-4 antibodies. A preferred clinical CTLA-4 antibody is human monoclonal antibody 10D1 (also referred to as MDX-010 and ipilimumab and available from Medarex, Inc., Bloomsbury, N.J.), disclosed in WO 01/14424.

As is known in the art, ipilimumab refers to an anti-CTLA-4 antibody, and is a fully human IgG1, antibody derived from transgenic mice having human genes encoding heavy and light chains to generate a functional human repertoire. ipilimumab can also be referred to by its CAS Registry No. 477202-00-9, and is disclosed as antibody 10DI in PCT Publication No. WOO 1/14424, incorporated herein by reference in its entirety and for all purposes. Specifically, ipilimumab describes a human monoclonal antibody or antigen-binding portion thereof that specifically binds to CTLA-4, comprising a light chain variable region and a heavy chain variable region having a light chain variable region comprised of SEQ ID NO:5, and comprising a heavy chain region comprised of SEQ ID NO:6. Pharmaceutical compositions of ipilimumab include all pharmaceutically acceptable compositions comprising ipilimumab and one or more diluents, vehicles and/or excipients. Examples of a pharmaceutical composition comprising ipilimumab are provided in PCT Publication No. WO2007/67959. Ipilimumab may be administered by I.V.

Light chain variable region for Ipilimumab:

(SEQ ID NO: 1)

EIVLTQSPGTLSLSPGERATLSCRASQSVGSSYLAWYQQKYGQAPRLLIY
GAFSRATGIPDRFSGSGSGTDFTLTISRLEPEDFAVYYCQQYGSSPWTFG
QGTKVEIK
Heavy chain variable region for Ipilimumab:

(SEQ ID NO: 2)

QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSS

As noted elsewhere herein, ALC slope may be useful as a predictive indicator of patient response to the administration of one or more anti-CTLA-4 antagonists, either alone or in combination with a peptide antigen (e.g., gp100), in addition to or in conjunction with an anti-proliferative agent disclosed herein. A non-limiting example of a peptide antigen would be a gp100 peptide comprising, or alternatively consisting of, the sequence selected from the group consisting of: IMDQVPFSV (SEQ ID NO:3), and YLEPGPVTV (SEQ ID NO:4). Such a peptide may be administered orally, or preferably at 1 mg emulsified in incomplete Freund's adjuvant (IFA) injected s.c. in one extremity, and 1 mg of either the same or a different peptide emulsified in IFA may be injected in another extremity.

Disorders for which the present invention may be useful for predicting patient responses to immunotherapy and/or co-stimulatory pathway modulation, for example, through the administration of ipilimumab, include, but are not limited to melanoma, primary melanoma, unresectable stage III or IV malignant melanoma, lung cancer, non-small cell lung cancer, small cell lung cancer, and prostate cancer.

Additional disorders for which the present invention may be useful for predicting patient responses to immunotherapy and/or co-stimulatory pathway modulation, for example, through the administration of ipilimumab, include, but are not limited to glioma, gastrointestinal cancer, renal cancer, ovarian cancer, liver cancer, colorectal cancer, endometrial cancer, kidney cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include uticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

The terms “treating”, “treatment” and “therapy” as used herein refer to curative therapy, prophylactic therapy, preventative therapy, and mitigating disease therapy.

The phrase “more aggressive dosing regimen” or “increased dosing frequency regimen”, as used herein refers to a dosing regimen that necessarily exceeds the basal and/or prescribed dosing regimen of a co-stimulatory pathway modulator, preferably ipilimumab, either due to an increased dosing frequency (about once a week, about bi-weekly, about once daily, about twice daily, etc.), increased or escalated dose (about 11, about 12, about 13, about 14, about 15, about 16, about 17, about 18, about 19, about 20, about 21, about 22, about 23, about 24, about 25, about 26, about 27, about 28, about 29, about 30, about 35, about 40 mg/ml), or the route of administration which may result in an increased, bio-available level of said co-stimulatory modulator.

It is to be understood this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, preferably ±5%, or ±1%, or as little as ±0.1% from the specified value, as such variations are appropriate to perform the disclosed methods.

Treatment regimens can be established based upon determining whether a patient exhibits either a positive or negative ALC slope subsequent to the administration of a co-stimulatory pathway modulator, such as ipilimumab, or other therapy described herein, such as chemotherapy. If a positive or negative ALC slope is detected in the sample from said patient, treatment regimens can be developed appropriately. For example, the presence of positive ALC slope may indicate said patient has an increased likelihood of achieving a clinical benefit and/or immune-related response to said co-stimulatory pathway modulator therapy, and thus warrants continuation of the prescribed therapeutic regimen. Alternatively, if a negative ALC slope is detected, it may indicate said patient has a decreased likelihood of achieving a clinical benefit and/or immune-related response to said co-stimulatory pathway modulator therapy, and thus may suggest that either higher doses of the co-stimulatory pathway modulator therapy should be administered or more aggressive dosing regimens or combination therapy are warranted. In one aspect, an increased dosing level of a co-stimulatory pathway modulator, such as ipilimumab, would be about 10, 20, 30, 40, 50, 60, 70, 80, 90, or 95% more than the typical co-stimulatory pathway modulator dose for a particular indication or individual (e.g., about 0.3 mg/kg, about 3 mg/kg, about 10 mg/kg, about 15 mg/kg, about 20 mg/kg, about 25 mg/kg, about 30 mg/kg), or about 1.5×, 2×, 2.5×, 3×, 3.5×, 4×, 4.5×, 5×, 6×, 7×, 8×, 9×, or 10× more co-stimulatory pathway modulator than the typical co-stimulatory pathway modulator dose for a particular indication or for individual.

A therapeutically effective amount of co-stimulatory pathway modulator, preferably ipilimumab, can be orally administered if it is a small molecule modulator, for example, or preferably injected into the patient. The actual dosage employed can be varied depending upon the requirements of the patient and the severity of the condition being treated, including consideration to the ALC slope. Determination of the proper starting dosage for a particular situation is within the skill of the art, though the assignment of a treatment regimen will benefit from taking into consideration the ALC slope. Nonetheless, it will be understood that the specific dose level and frequency of dosing for any particular patient can be varied and will depend upon a variety of factors including the activity of the specific compound employed, the metabolic stability and length of action of that compound, the species, age, body weight, general health, sex and diet of the patient, the mode and time of administration, rate of excretion, drug combination, and severity of the particular condition. Preferred patients for treatment include animals, most preferably mammalian species such as humans, and domestic animals such as dogs, cats, and the like, patient to cancer.

The terms “combination” and “combinations” as used herein refer to a combination of a co-stimulatory pathway modulator, preferably an agonist, with another co-stimulatory pathway modulator, preferably an agonist (i.e., immunostimulant), PROVENGE®, a tubulin stabilizing agent (e.g., pacitaxol, epothilone, taxane, etc.), Bevacizumab, IXEMPRA™, Dacarbazine, PARAPLATIN®, Docetaxel, one or more peptide vaccines, MDX-1379 Melanoma Peptide Vaccine, one or more gp100 peptide vaccine, fowlpox-PSA-TRICOM™ vaccine, vaccinia-PSA-TRICOM™ vaccine, MART-1 antigen, sargramostim, ticilimumab, Combination Androgen Ablative Therapy; the combination of ipilimumab and another co-stimulatory pathway modulator; combination of ipilimumab and a tubulin stabilizing agent (e.g., pacitaxol, epothilone, taxane, etc.); combination of ipilimumab and IXEMPRA™ the combination of ipilimumab with Dacarbazine, the combination of ipilimumab with PARAPLATIN®, the combination of ipilimumab with Docetaxel, the combination of ipilimumab with one or more peptide vaccines, the combination of ipilimumab with MDX-1379 Melanoma Peptide Vaccine, the combination of ipilimumab with one or more gp100 peptide vaccine, the combination of ipilimumab with fowlpox-PSA-TRICOM™ vaccine, the combination of ipilimumab with vaccinia-PSA-TRICOM™ vaccine, the combination of ipilimumab with MART-1 antigen, the combination of ipilimumab with sargramostim, the combination of ipilimumab with ticilimumab, and/or the combination of ipilimumab with Combination Androgen Ablative Therapy. The combinations of the present invention may also be used in conjunction with other well known therapies that are selected for their particular usefulness against the condition that is being treated. Such combinations may provide therapeutic options to those patients who present with a negative ALC slope during the ALC slope interval.

In another embodiment of the present invention, combination between a co-stimulatory pathway modulator and at least one other agent may comprise one or more of the following combinations: ipilimumab and Taxol and Paraplatin (concurrent administration); ipilimumab and Taxol and Paraplatin (sequential administration); ipilimumab and Dacarbazine; ipilimumab and Bevacizumab; ipilimumab and Budesonide; ipilimumab and an inhibitor of CD137; and ipilimumab and steroids (corticosteroids and the like).

ALC slope may be useful as a predictive indicator of patient response to other co-stimulatory pathway modulators alone, or response to co-stimulatory pathway modulators in combination with other co-stimulatory pathway modulators disclosed herein, or response to combination with other compounds disclosed herein, which include, but are not limited to, the following: agatolimod, belatacept, blinatumomab, CD40 ligand, anti-B7-1 antibody, anti-B7-2 antibody, anti-B7-H4 antibody, AG4263, eritoran, anti-CD137 monoclonal antibodies, anti-OX40 antibody, ISF-154, and SGN-70.

A variety of chemotherapeutics are known in the art, some of which are described herein. One type of chemotherapeutic is referred to as a metal coordination complex. It is believed this type of chemotherapeutic forms predominantly inter-strand DNA cross links in the nuclei of cells, thereby preventing cellular replication. As a result, tumor growth is initially repressed, and then reversed. Another type of chemotherapeutic is referred to as an alkylating agent. These compounds function by inserting foreign compositions or molecules into the DNA of dividing cancer cells. As a result of these foreign moieties, the normal functions of cancer cells are disrupted and proliferation is prevented. Another type of chemotherapeutic is an antineoplastic agent. This type of agent prevents, kills, or blocks the growth and spread of cancer cells. Still other types of anticancer agents include nonsteroidal aromastase inhibitors, bifunctional alkylating agents, etc.

Immunotherapy, in combination with chemotherapy, is a novel approach for the treatment of cancer which combines the effects of agents that directly attack tumor cells producing tumor cell necrosis or apoptosis, and agents that modulate host immune responses to the tumor. Chemotherapeutic agents could enhance the effect of immunotherapy by generating tumor antigens to be presented by antigen-presenting cells creating a “polyvalent” tumor cell vaccine, and by distorting the tumor architecture, thus facilitating the penetration of the immunotherapeutic agents as well as the expanded immune population.

ALC slope may be useful as a predictive indicator of patient response to microtubule-stabilizing agents, such as ixabepilone (IXEMPRA™) and paclitaxel (TAXOL®), which commonly are used for the treatment of many types of cancer and represent an attractive class of agents to combine with CTLA-4 blockade.

The phrase “microtubulin modulating agent” is meant to refer to agents that either stabilize microtubulin or destabilize microtubulin synthesis and/or polymerization.

One microtubulin modulating agent is paclitaxel (marketed as TAXOL®), which is known to cause mitotic abnormalities and arrest, and promotes microtubule assembly into calcium-stable aggregated structures resulting in inhibition of cell replication.

Epothilones mimic the biological effects of TAXOL®, (Bollag et al., Cancer Res., 55:2325-2333 (1995), and in competition studies act as competitive inhibitors of TAXOL® binding to microtubules. However, epothilones enjoy a significant advantage over TAXOL® in that epothilones exhibit a much lower drop in potency compared to TAXOL® against a multiple drug-resistant cell line (Bollag et al. (1995)). Furthermore, epothilones are considerably less efficiently exported from the cells by P-glycoprotein than is TAXOL® (Gerth et al. (1996)). Additional examples of epothilones are provided in co-owned, PCT Application No. PCT/US2009/030291, filed Jan. 7, 2009, which is hereby incorporated by reference herein in its entirety for all purposes.

Ixabepilone is a semi-synthetic lactam analogue of patupilone that binds to tubulin and promotes tubulin polymerisation and microtubule stabilisation, thereby arresting cells in the G2/M phase of the cell cycle and inducing tumour cell apoptosis.

Additional examples of microtubule modulating agents useful in combination with immunotherapy include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (TAXOL®, NSC 125973), TAXOL® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, epothilone C, epothilone D, desoxyepothilone A, desoxyepothilone B, [1S-[1R*,3R*(E),7R*,10S*,11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo[14.1.0]heptadecane-5,9-dione (disclosed in U.S. Pat. No. 6,262,094, issued Jul. 17, 2001), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. Ser. No. 09/506,481 filed on Feb. 17, 2000, and examples 7 and 8 herein), and derivatives thereof; and other microtubule-disruptor agents. Additional antineoplastic agents include, discodermolide (see Service, Science, 274:2009 (1996)) estramustine, nocodazole, MAP4, and the like. Examples of such agents are also described in the scientific and patent literature, see, e.g., Bulinski, J. Cell Sci., 110:3055-3064 (1997); Panda, Proc. Natl. Acad. Sci. USA, 94:10560-10564 (1997); Muhlradt, Cancer Res., 57:3344-3346 (1997); Nicolaou, Nature, 387:268-272 (1997); Vasquez, Mol. Biol. Cell., 8:973-985 (1997); Panda, J. Biol. Chem., 271:29807-29812 (1996).

The following sets forth preferred therapeutic combinations and exemplary dosages for use in the methods of the present invention.

| | DOSAGE |
|---|---|
| THERAPEUTIC COMBINATION | mg/m² (per dose) |
| Ixabepilone + | 1-500 mg/m2 |
| anti-CTLA-4 Antibody | 0.1-25 mg/kg |
| Paclitaxel + | 40-250 mg/m2 |
| anti-CTLA-4 Antibody | 0.1-25 mg/kg |

While this table provides exemplary dosage ranges of co-stimulatory pathway modulators and certain anticancer agents of the invention, when formulating the pharmaceutical compositions of the invention the clinician may utilize preferred dosages as warranted by the condition of the patient being treated. For example, ixabepilone may preferably be administered at about 40 mg/m2 every 3 weeks. Paclitaxel may preferably be administered at about 135-175 mg/m2 every three weeks.

The anti-CTLA-4 antibody may preferably be administered at about 0.3-10 mg/kg, or the maximum tolerated dose. In an embodiment of the invention, a dosage of CTLA-4 antibody is administered about every three weeks. Alternatively, the CTLA-4 antibody may be administered by an escalating dosage regimen including administering a first dosage of CTLA-4 antibody at about 3 mg/kg, a second dosage of CTLA-4 antibody at about 5 mg/kg, and a third dosage of CTLA-4 antibody at about 9 mg/kg.

In another specific embodiment, the escalating dosage regimen includes administering a first dosage of CTLA-4 antibody at about 5 mg/kg and a second dosage of CTLA-4 antibody at about 9 mg/kg.

Further, the present invention provides an escalating dosage regimen, which includes administering an increasing dosage of CTLA-4 antibody about every six weeks.

In an aspect of the present invention, a stepwise escalating dosage regimen is provided, which includes administering a first CTLA-4 antibody dosage of about 3 mg/kg, a second CTLA-4 antibody dosage of about 3 mg/kg, a third CTLA-4 antibody dosage of about 5 mg/kg, a fourth CTLA-4 antibody dosage of about 5 mg/kg, and a fifth CTLA-4 antibody dosage of about 9 mg/kg. In another aspect of the present invention, a stepwise escalating dosage regimen is provided, which includes administering a first dosage of 5 mg/kg, a second dosage of 5 mg/kg, and a third dosage of 9 mg/kg.

The actual dosage employed may be varied depending upon the requirements of the patient and the severity of the condition being treated, which may be determined by consideration of the ALC slope in accordance with the present invention. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In practicing the many aspects of the invention herein, biological samples can be selected preferably from blood, blood cells (red blood cells or white blood cells). Cells from a sample can be used, or a lysate of a cell sample can be used. In certain embodiments, the biological sample comprises blood cells.

Pharmaceutical compositions for use in the present invention can include compositions comprising one or a combination of co-stimulatory pathway modulators in an effective amount to achieve the intended purpose. A therapeutically effective dose refers to that amount of active ingredient which ameliorates the symptoms or condition, and should take into consideration the ALC slope in accordance with the present invention. Therapeutic efficacy and toxicity in humans can be predicted by standard pharmaceutical procedures in cell cultures or experimental animals, for example the ED50 (the dose therapeutically effective in 50% of the population) and LD50 (the dose lethal to 50% of the population).

A “therapeutically effective amount” of a modulator of the co-stimulatory pathway can be a function of whether a patient exhibits a positive or negative ALC slope. A therapeutically relevant dose of a co-stimulatory pathway modulator for patients having a negative ALC slope, for example, could range anywhere from 1 to 14 fold or more higher than the typical dose. Accordingly, therapeutically relevant doses of a co-stimulatory pathway modulator, such as ipilimumab, for any disorder disclosed herein, preferably melanoma, can be, for example, about 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 fold higher than the prescribed or standard dose. Alternatively, therapeutically relevant doses of a co-stimulatory pathway modulator, such as ipilimumab, can be, for example, about 1.0×, about 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01× of the prescribed dose for individuals exhibiting a positive ALC slope.

The present invention provides methods of determining responsiveness of an individual having a disorder to a certain treatment regimen and methods of treating an individual having a disorder based upon determining whether a patient exhibits a positive or negative ALC slope subsequent to the administration of said treatment regimen for a given time interval.

Disorders for which ALC slope may be useful as a predictive indicator of patient response beyond merely melanoma, prostate cancer, and lung cancer, for example, also include leukemias, including, for example, chronic myeloid leukemia (CML), acute lymphoblastic leukemia, and Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ ALL, AML, imatinib-resistant CML, imatinib-intolerant CML, accelerated CML, lymphoid blast phase CML.

The terms “cancer”, “cancerous”, or “malignant” refer to or describe the physiological condition in mammals, or other organisms, that is typically characterized by unregulated cell growth. Examples of cancer include, for example, solid tumors, melanoma, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

A “solid tumor” includes, for example, sarcoma, melanoma, colon carcinoma, breast carcinoma, prostate carcinoma, or other solid tumor cancer.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ ALL).

### Antibodies

ALC slope may be useful as a predictive indicator of patient response to antibodies that can specifically bind to co-stimulatory pathway polypeptides, such as CTLA-4, CD28, CD80, and CD86. The term “antibody” is used in the broadest sense and specifically covers monoclonal antibodies, polyclonal antibodies, antibody compositions with polyepitopic specificity, bispecific antibodies, diabodies, chimeric, single-chain, and humanized antibodies, as well as antibody fragments (e.g., Fab, F(ab′)₂, and Fv), so long as they exhibit the desired biological activity. Antibodies can be labeled for use in biological assays (e.g., radioisotope labels, fluorescent labels) to aid in detection of the antibody.

Antibodies that bind to co-stimulatory pathway polypeptides can be prepared using, for example, intact polypeptides or fragments containing small peptides of interest, which can be prepared recombinantly for use as the immunizing antigen. The polypeptide or oligopeptide used to immunize an animal can be derived from the translation of RNA or synthesized chemically, and can be conjugated to a carrier protein, if desired. Commonly used carriers that are chemically coupled to peptides include, for example, bovine serum albumin (BSA), keyhole limpet hemocyanin (KLH), and thyroglobulin. The coupled peptide is then used to immunize the animal (e.g., a mouse, a rat, or a rabbit).

The term “antigenic determinant” refers to that portion of a molecule that makes contact with a particular antibody (i.e., an epitope). When a protein or fragment of a protein is used to immunize a host animal, numerous regions of the protein can induce the production of antibodies that bind specifically to a given region or three-dimensional structure on the protein; each of these regions or structures is referred to as an antigenic determinant. An antigenic determinant can compete with the intact antigen (i.e., the immunogen used to elicit the immune response) for binding to an antibody.

The phrase “specifically binds to” refers to a binding reaction that is determinative of the presence of a target in the presence of a heterogeneous population of other biologics. Thus, under designated assay conditions, the specified binding region binds preferentially to a particular target and does not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice background signal or noise and more typically more than 10 times background. For purposes of the present invention, compounds, for example small molecules, can be considered for their ability to specifically bind to co-stimulatory pathway polypeptides described herein.

### Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise a means for performing an absolute lymphocyte count on a patient sample and/or instructions for interpreting the ALC value obtained. Another example of a container means can comprise one or more vials containing a pharmaceutically acceptable amount of a co-stimulatory pathway modulator.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

Kits useful in practicing therapeutic methods disclosed herein can also contain a compound that is capable of inhibiting the co-stimulatory pathway. Specifically contemplated by the invention is a kit comprising an anti-CTLA-4 antibody, either alone or in combination with another immunotherapy agent, such as PROVENGE®; a tubulin stabilizing agent (e.g., pacitaxol, epothilone, taxane, etc.); and/or a second co-stimulatory pathway modulator, such as, tremelimumab. In addition, contemplated by the invention is a kit comprising an increased dose and/or dosing frequency regimen of a co-stimulatory pathway modulator, and any other combination or dosing regimen comprising a tubulin stabilizing agent (e.g., pacitaxol, epothilone, taxane, etc.); and/or a second co-stimulatory pathway modulator, such as, tremelimumab.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope.

### REFERENCES

1. Margolin K. Moving forward with immunotherapy: the rationale for anti-CTLA-4 therapy in melanoma. Comm Oncol, 2008;5: 367-74.

2. Melero I, Hervas-Stubbs S, Glennie M, Pardoll D M, Chen L. Immunostimulatory monoclonal antibodies for cancer therapy. Nat Rev Cancer, 2007; 7:95-106.

3. Wolchok J D, Saenger Y. The mechanism of anti-CTLA-4 activity and the negative regulation of T-cell activation. The Oncologist 2008; 13 (suppl. 4):2-9.

4. Quezada S A, Peggs K S, Curran M A, Allison J P. CTLA-4 blockade and GM-CSF combination immunotherapy alters the intratumor balance of effector and regulatory T cells. J Clin Invest, 2006; 116:1935-45.

5. Weber J S, O'Day S, Urba W, et al. Phase I/II study of ipilimumab for patients with metastatic melanoma. J Clin Oncol, 2008; 26:5950-56.

6. Weber J. Ipilimumab: controversies in its development, utility and autoimmune adverse events. Cancer Immunol. Immunother, 2009; 58:823-30.

7. Cox M C, Nofroni I, Ruco L. et al. Low absolute lymphocyte count is a poor prognostic factor in diffuse-large-B-cell-lymphoma. Leuk Lymphoma, 2008; 49: 1745-51.

8. De Angulo G, Hernandez M, Morales-Arias J, et al. Early lymphocyte recovery as a prognostic indicator for high-risk Ewing sarcoma. J Pediatr Hematol Oncol, 2007; 29:48-52.

9. De Angulo G, Yuen C, Palla S L, Anderson P M, Zweidler-McKay P A. Absolute lymphocyte count is a novel prognostic indicator in ALL and AML: implications for risk stratification and future studies. Cancer 2008, 112:407-15.

10. Ege H, Gertz M A, Markovic S N. et al. Prediction of survival using absolute lymphocyte count for newly diagnosed patients with multiple myeloma: a retrospective study. Br J Haematol, 2008; 141:792-98.

11. Claude L, Perol D, Ray-Coquard I, et al. Lymphopenia: a new independent prognostic factor for survival in patients treated with whole brain radiotherapy for brain metastases from breast carcinoma. Radiother Oncol 2005; 76:334-39.

12. Maltoni M, Caraceni A, Brunelli C, et al. Prognostic factors in advanced cancer patients: evidence-based clinical recommendations—a study by the Steering Committee of the European Association for Palliative Care, J Clin Oncol, 2005; 23:6240-48.

13. Hodi F S, Hoos A, Ibrahim R, et al. Novel efficacy criteria for antitumor response to immunotherapy using the example of ipilimumab, an anti-CTLA-4 monoclonal antibody. J Clin Oncol, 2008; 26(19s):abstr 3008.

14. Hamid O, Chin K, Li J, et al. Dose effect of ipilimumab in patients with advanced melanoma: results from a phase II, randomized, dose-ranging study. J Clin Oncol, 2008, 26(19s):abstr 9025.

15. O'Day S J, Ibrahim R, DePril V, et al. Efficacy and safety of ipilimumab induction and maintenance dosing in patients with advanced melanoma who progressed on one or more prior therapies. J Clin Oncol, 2008; 26(19s):abstr 9021.

16. Rosenberg S A, Sherry R M, Morton K E, et al. Tumor progression can occur despite the induction of very high levels of self/tumor antigen-specific CD8⁺ T cells in patients with melanoma. J Immunol, 2005; 175:6169-76.

17. Behl D, Ristow K, Markovic S N, et al. Absolute lymphocyte count predicts therapeutic efficacy of rituximab therapy in follicular lymphomas. Br J Haematol, 2007; 137:409-15.

18. Oki Y, Yamamoto K, Kato H, et al. Low absolute lymphocyte count is a poor prognostic marker in patients with diffuse large B-cell lymphoma and suggests patients' survival benefit from rituximab. Eur J Haematol, 2008; 81:448-53.

19. Ridolfi L, Ridolfi R. Preliminary experiences of intralesional immunotherapy in cutaneous metastatic melanoma. Hepatogastroenterology, 2002; 49:335-39.

20. Viganó A, Bruera E, Jhangri G S, Newman S C, Fields A L, Suarez-Almazor M E. Clinical survival predictors in patients with advanced cancer. Arch Intern Med, 2000; 160:861-68.

21. Dai, G., Pfister, M., Blackwood-Chirchirm, A. & Roy, A. Importance of characterizing determinants of variability in exposure: application to dasatinib in subjects with chronic myeloid leukemia, J. Clin. Pharmacol, 2008, 48:1254-1269.

### Examples

### Example 1

### Methods Used to Associate Absolute Lymphocyte Count with Beneficial Response to Costimulatory Pathway Inhibition for Three Phase II Studies

CTLA-4 is a negative regulator of the activation of T cell lymphocytes. By blocking CTLA-4, ipilimumab activates the T cell lymphocyte leading to increased anti-tumor activity and T cell proliferation. Across three phase II studies in patients with melanoma, circulating lymphocytes (absolute lymphocyte count, ALC) were measured at baseline and through the first 12 weeks after first dose of ipilimumab (induction period dosing). The change in ALC over time (ALC slope) was measured. Ipilimumab induced a dose-dependent increase in the circulating lymphocytes with 10 mg/kg inducing greater average rates of increase (slopes) than did 3 or 0.3 mg/kg. The ALC is a routine and clinically accepted blood cell parameter that is measured by oncologists and labs prior to therapy administration. It is believed the present invention is the first application of using ALC slope as a factor to predict clinical benefit to a therapeutic regimen.

The results demonstrate that no patient with a decrease in ALC during the induction dosing period experienced Clinical Benefit (defined by objective response or prolonged stable disease). Patients who did have Clinical Benefit had, on average, a higher rate of increase in ALC over time, than did patients without Clinical Benefit. Therefore, the inventors propose that the change in circulating immune cells (i.e., ALC for ipilimumab or other lymphocyte activators) over time may be used to predict Clinical Benefit and possibly increased survival. A threshold rate of change in ALC over time may be used to identify patients that are likely or unlikely to experience Clinical Benefit. Such a biomarker could be used for negative enrichment (i.e., recommend possible cessation of treatment on account of such patients having a lower likelihood of achieving a beneficial response) or positive enrichment (i.e., recommend continuation of treatment on account of such patients having a higher likelihood of achieving a beneficial response).

### Methods

Data were collected from patients with unresectable stage III or IV melanoma who participated in three phase II clinical trials: CA184-008 (NCT00289627) was a multicenter, single-arm study of ipilimumab monotherapy in previously treated patients; CA184-022 (NCT00289640) was a randomized, double-blind, multi-center, fixed dose study of multiple doses of ipilimumab monotherapy in previously treated patients; CA184-007 (NCT00135408) was a randomized, double-blind, placebo-controlled study comparing the safety of ipilimumab administered with or without prophylactic oral budesonide in untreated and previously treated patients. Full details on these clinical details are available on the U.S. Government's Clinicaltrials web site. All protocols were approved by an Institutional Review Board or Independent Ethics Committee; all studies were carried out in accordance with the ethical principles of the Declaration of Helsinki and the International Conference on Harmonization of Good Clinical Practice.

Ipilimumab was administered at 0.3, 3, or 10 mg/kg as a 90-minute outpatient intravenous infusion every three weeks for four separate doses (weeks 1, 4, 7, and 10) during the induction phase. Patients with progressive disease (PD) before week 12 (according to modified World Health Organization criteria; heretofore referred to as mWHO criteria) continued receiving ipilimumab provided they did not experience rapid clinical deterioration. Eligible patients could continue to receive ipilimumab every 12 weeks beginning at week 24 (maintenance phase).

The antitumor response of ipilimumab in clinical studies was evaluated by an independent review committee (IRC) using mWHO criteria. The first scheduled tumor assessment was at week 12, and any assessment of CR or PR was to be confirmed at least four weeks after response criteria were first met. Response-evaluable patients were classified into a response category by presence or absence of clinical benefit. Response Category (RESPONSE) according to the mWHO criteria, was determined as follows: where BORIRC=Best Overall Response as assessed by an independent review committee (IRC); CR=Complete Response; PR=Partial Response; SD=Stable Disease; and PD=Progressive Disease. If BORIRC equals any value not in the CR, PR, SD, or PD category, RESPONSE=“Unknown”. If BORIRC equals CR or PR, RESPONSE=“Benefit”. If BORIRC equals PD, RESPONSE=“Non-benefit”. If BORIRC equals SD, then response was assigned based upon one of the following criteria: (i) If study day for end of SD is missing, RESPONSE=“Unknown”; (ii) If study day for end of SD>=168, RESPONSE=“Benefit” (i.e., prolonged SD); (iii) If study day for end of SD<168 and censoring status for SD duration is missing, RESPONSE=“Unknown”; (iv) If study day for end of SD<168 and SD duration is not censored, RESPONSE=“Non-benefit”; (v) If study day for end of SD<168 and SD duration is censored and death date is not missing and death date<168, RESPONSE=“Non-benefit”; and (vi) If study day for end of SD<168 and SD duration is censored and death date is either missing or >=168, RESPONSE =“Unknown”.

Novel immune-related Response Criteria (irRC) were also used to evaluate antitumor responses, which capture the unique antitumor response patterns that have been observed with ipilimumab in clinical studies (Hodi et al., J Clin Oncol 2008;26(19s):abstr 3008). The irRC determine total tumor burden as the sum of the products of the two largest perpendicular diameters of measurable index (baseline) lesions and measurable new lesions, based on IRC measurements (Wolchok et al., manuscript in preparation). Determination of irBOR is therefore based on a reduction in total tumor burden, regardless of any initial increase in tumor burden and/or the appearance of new lesions which may characterize a patient as PD by mWHO criteria.

The irRC endpoints were defined as follows: decrease of total tumor burden from baseline by 100%, irCR; decrease from baseline of ≧50% in total tumor burden, irPR; decrease in total tumor burden of ≧25% but less than the 50%, irSD. A patient with new lesions that outweighed any decrease in the size of existing index lesions, which resulted in a ≧95% increase in total tumor burden, was considered an irPD. A composite efficacy endpoint, immune-related Clinical Activity (irCA), was used to describe the total measurable antitumor activity of ipilimumab (irCR, irPR, or irSD).

Serum samples for pharmacokinetic analyses were collected according to the following schedule: pre-dose on study days 1 and 43; 90-min post-infusion on study days 1 and 43; between 3-7 days post-dose on days 45-49 and between 10-15 days post-dose on days 52-57. In all three phase II studies, ipilimumab serum concentrations were measured using a quantitative enzyme-linked immunosorbent assay developed by Bristol-Myers Squibb. A non-compartmental serum pharmacokinetic analysis of ipilimumab was derived from serum concentration versus time data by a validated pharmacokinetic analysis program (Kinetica™ Basic Version 4.02, InnaPhase Corporation, 2002).

Peripheral ALC from routine safety labs were collected from 482 patients across the three phase II studies. Estimated mean ALC was obtained from an extended linear model fit by REML, with spatial exponential within-patient correlation structure (Euclidean distance), and within-patient variances inversely proportional to the number of ALC measures on a given day. Fixed effects were dose, time, and an additive interaction between dose and time. The change in ALC over time was modeled using splines with a knot at 0: linear before 0 and cubic after. This allowed the slope before first dose to possibly differ from the slope after first dose.

Two patients were excluded from all analyses presented here: one patient with an uncertain date of first dose, and one patient with an extremely large increase in ALC over time.

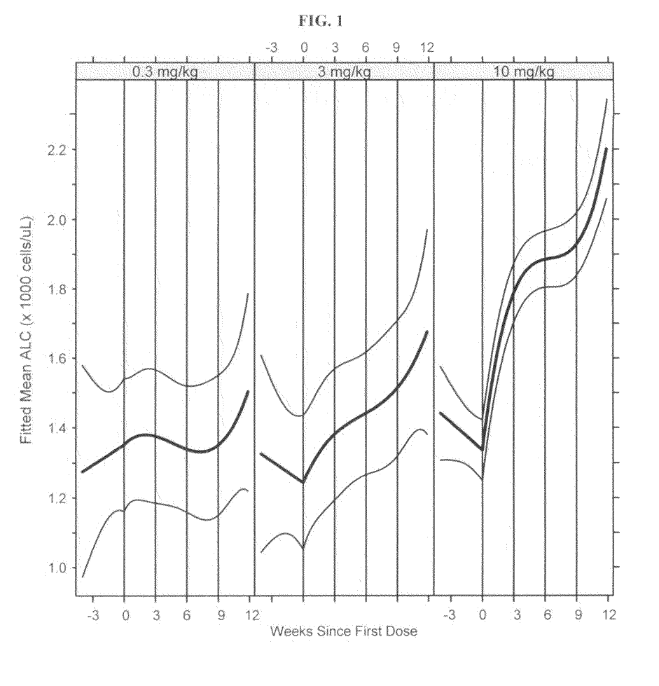

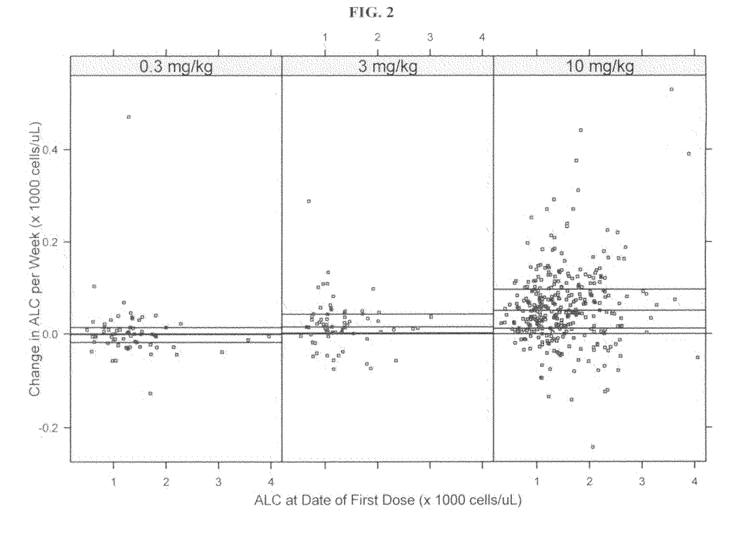

Modeling details are outlined in the legends of FIGS. 1, 2, 3, 4, 5 and 6.

### Exposure-Response (E-R) Analysis for BOR of CR or PR and irCA

Ipilimumab exposure in patients with advanced melanoma was characterized by a nonlinear mixed-effects compartmental pharmacokinetic model (population PK model). Ipilimumab serum concentration-time data were characterized by a linear, two-compartment, zero-order IV infusion model with first-order elimination.

Individual estimates of Cminₛₛ were defined as the steady-state concentrations at day 21 (3 weeks) post-infusion and obtained from predictions of steady-state observations using the MAP Bayesian estimates of all PK parameters. E-R relationships were characterized for IRC-determined BOR of CR or PR by mWHO criteria and irCA. The latter is a composite efficacy endpoint derived from irRC, such that irCA responders are patients who achieved a best overall ir-response of irCR, irPR, or late response (irCR or irPR or irSD after tumor progression), or irSD with ≧25% reduction in total tumor burden.

The E-R relationship for both BOR and irCA were characterized by logistic regression models that related ipilimumab Cminₛₛ to the probability of BOR or irCA. The existence and functional form of E-R relationship was established by a base model and the effect of the following covariates was assessed: body weight, age, gender, LDH, ECOG status, concomitant budesonide, metastatic stage, HLA.A2*201 genotype, prior immunotherapy, prior IL-2 therapy, and prior systemic anti-cancer therapy. The magnitude and statistical significance of each covariate was assessed by a forward inclusion and backward elimination method.

Covariates that were significant at 0.05 level by the log-likelihood ratio test (LRT) in the screening step were included in a covariate model, which was simplified by backward elimination to only retain covariates that were significant at 0.001 level by the LRT (Dai et al., J. Clin. Pharmacol. 48, 1254-1269 (2008)). The confidence interval of model predicted probability were obtained by bootstrap (n=500) and model evaluation was performed with predictive check. The final model was evaluated by comparing the observed proportion of patients achieving BOR or irCA with the 90% prediction interval of the proportion, for each dose group in the E-R dataset. The 90% prediction interval was obtained by 500 simulations with the final E-R model. All analysis was performed using the NONMEM computer program in Linux (Version VI, GloboMax, Hanover, Md.).

### Results

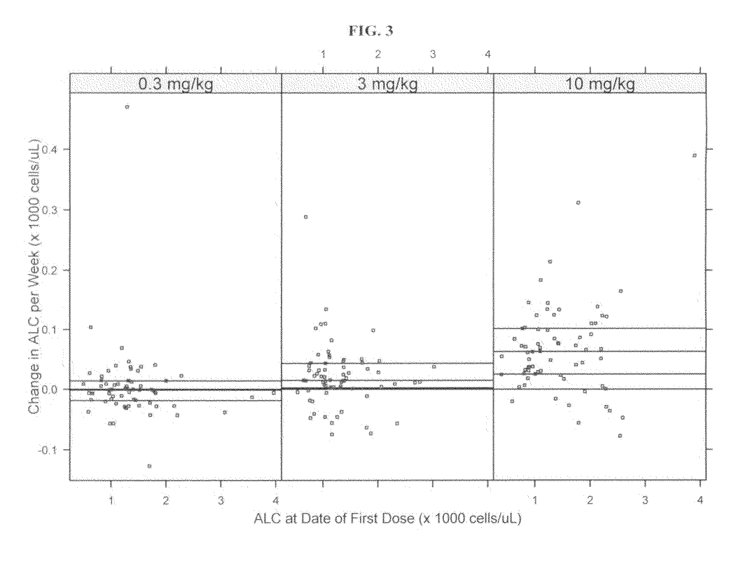

For patients combined over studies CA184-007, -008, and -022, 10 mg/kg ipilimumab induced a greater average rate of increase in ALC than did 3.0 mg/kg or 0.3 mg/kg (FIGS. 1 and 2, Table 1). The difference in mean slope between the 0.3 mg/kg and 10 mg/kg groups was statistically significant (test of time-by-dose interaction from the extended linear spline model shown in FIG. 2: t=2.10, p=0.036; a similar test from an extended linear model with cubic time effect but no splines gave t=4.09, p=4.4×10e-5).

All patients in the 0.3 mg/kg and 3 mg/kg groups were from study CA184-022. Thus, the trend of increasing ALC slope with increasing dose could potentially reflect an unknown difference among the studies, rather than dose per se. However, this trend also was present for patients from study CA184-022 alone (FIG. 3). This argues against the potential alternative explanation, suggesting that the association between ALC slope and ipilimumab dose seen for the 3 studies combined did not result from the difference in distribution of doses among studies.

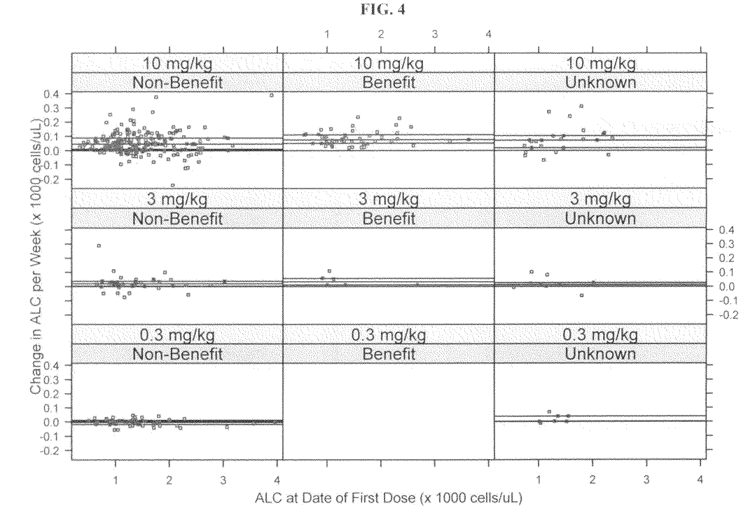

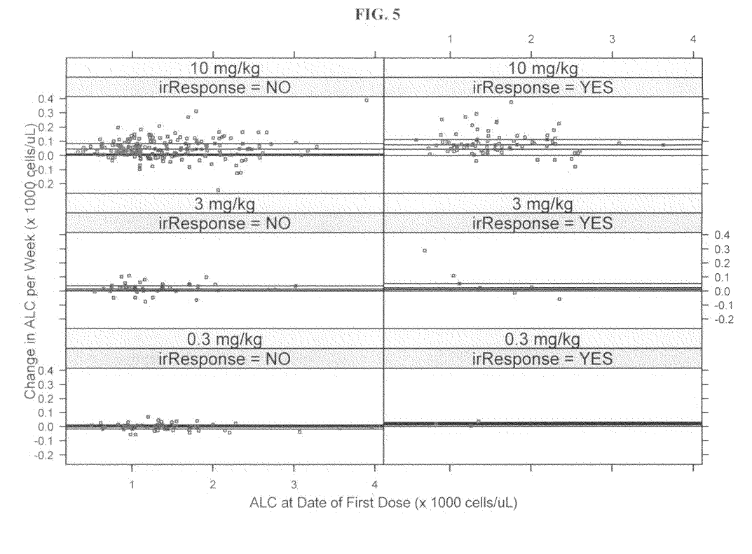

The primary endpoint of anti-tumor activity was based on the definition of Clinical Benefit using the modified WHO (mWHO) criteria. No patient with a negative ALC slope—that is, a decrease in ALC over the induction dosing period—experienced Clinical Benefit (FIG. 4, Table 1). Patients who did have Clinical Benefit had, on average, a higher rate of increase over time (slope), than did patients without Clinical Benefit (FIG. 4, Table 1). The difference in mean slope between the Benefit and Non-Benefit groups for patients who received 10 mg/kg ipilimumab was highly statistically significant (Welch modified 2-sample t-test of the per-patient slope estimates: t=3.52, df=110, p=6×10e-04). The similar difference for patients who received 3 mg/kg ipilimumab was not statistically significant.

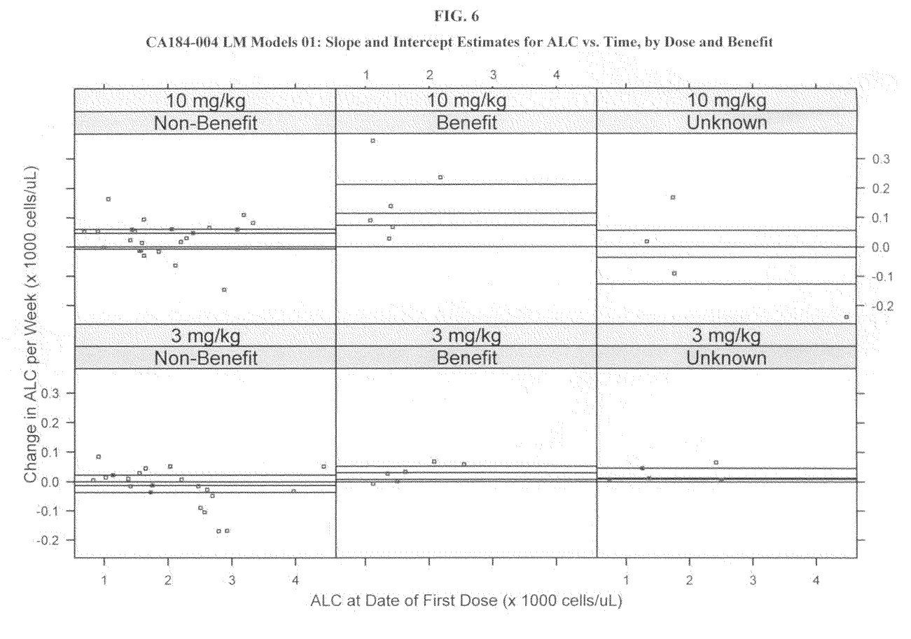

irResponse is an exploratory measure of the anti-tumor activity of ipilimumab and has not yet been validated. As observed for Clinical Benefit, patients with an irResponse had, on average, a higher rate of increase over time (slope), than did patients who did not (FIG. 5, Table 1). The difference in mean slope between the irResponse categories for patients who received 10 mg/kg ipilimumab was highly statistically significant (Welch modified 2-sample t-test of the per-patient slope estimates: t=3.69, df=138, p=3×10e-04). The similar difference for patients who received 3 mg/kg ipilimumab was not statistically significant. Few (7/89) patients with negative ALC slopes—that is, a decrease in ALC over the induction dosing period—experienced immune-related response (irResponse) (FIG. 5, Table 1). It is believed these individuals may have achieved or were about to achieve a positive slope, but the positive inclination of the slope was not observed because the analysis was limited to a 12-week period. Longer analysis periods will be assessed to determine whether such individuals did in fact achieve a positive slope.

Table 1. Provides a summary of Per-Patient Absolute Lymphocyte Count (ALC) Change per Week (Slope) over the induction dosing period, for all patients with known date of first dose, at least 1 post-first-dose ALC value, and at least 2 ALC values between study days −28 and 84, inclusive (n=462). N=number of patients in group, SD=Standard Deviation, Total=All patients in data set, Benefit=Patients with IRC BOR of CR or PR, or prolonged SD, with a duration at least 24 weeks from date of first dose; Non-Benefit =Patients with IRC BOR of PD, or non-prolonged SD; Unknown=Patients not in Benefit or Non-Benefit groups. All groups except “Total” include only response-evaluable patients.

TABLE 1

| | | | | | | Fraction |
|---|---|---|---|---|---|---|
| | | | | Mean | SD | Negative |
| Study | Dose | Group | N | Slope | Slope | Slope |
| Pooled | 0.3 mg/kg | Total | 64 | 0.005 | 0.068 | 0.58 |
| (007, 008, 022) | | Benefit | 0 | N/A | N/A | N/A |
| | | Non-Benefit | 47 | −0.005 | 0.024 | 0.60 |
| | | Unknown | 7 | 0.019 | 0.029 | 0.43 |
| | | irResponse = YES | 3 | 0.020 | 0.018 | 0.00 |
| | | irResponse = NO | 51 | −0.003 | 0.026 | 0.61 |
| | 3.0 mg/kg | Total | 69 | 0.021 | 0.054 | 0.23 |
| | | Benefit | 6 | 0.043 | 0.039 | 0.00 |
| | | Non-Benefit | 39 | 0.023 | 0.057 | 0.21 |
| | | Unknown | 9 | 0.022 | 0.048 | 0.22 |
| | | irResponse = YES | 9 | 0.051 | 0.100 | 0.22 |
| | | irResponse = NO | 45 | 0.020 | 0.039 | 0.18 |
| | 10.0 mg/kg | Total | 329 | 0.059 | 0.083 | 0.18 |
| | | Benefit | 49 | 0.086 | 0.051 | 0.00 |
| | | Non-Benefit | 197 | 0.054 | 0.077 | 0.18 |
| | | Unknown | 25 | 0.077 | 0.091 | 0.20 |
| | | irResponse = YES | 80 | 0.088 | 0.078 | 0.06 |
| | | irResponse = NO | 191 | 0.051 | 0.072 | 0.19 |

### Example 2

### Methods Used to Associate Absolute Lymphocyte Count with Beneficial Response to Costimulatory Pathway Inhibition for Study CA184-004

The relationship between ALC slope and patient response to ipilimumab was further assessed in an additional phase II study, CA184-004.

Data were collected from patients with unresectable stage III or IV melanoma who participated in the phase II clinical trial: CA184-004. CA184-004 (NCT00261365) was a randomized, double-blind, multi-center, fixed dose study of multiple doses of ipilimumab monotherapy in previously treated patients. Full details on these clinical details are available at the U.S. Government's Clinicaltrials website. All protocols were approved by an Institutional Review Board or Independent Ethics Committee; all studies were carried out in accordance with the ethical principles of the Declaration of Helsinki and the International Conference on Harmonization of Good Clinical Practice.

### Methods

Methods used were performed according to the methods outlined in Example 1 herein.

### 004 Results and Overall Combined Studies Results

In analysis of the independent data from study CA184-004 (n=65), the inventors confirmed that ALC slope is associated with clinical benefit (FIG. 6). Across all 4 studies, the percent of patients with a negative ALC slope was 58% (37/64) at 0.3 mg/kg [study CA184-022], 28% (29/104) at 3 mg/kg [studies CA184-022 and -004], and 19% (71/365) at 10 mg/kg [studies CA184-007, -008, -022, and -004]. In the 0.3 and 3 mg/kg groups, the relatively high percentage of patients with a negative ALC slope was most likely due to insufficient ipilimumab exposure.

A summary of the results obtained for the analysis of the CA184-004 study is shown in Table 2. Analysis of the combined results shown in Tables 1 and 2 shows a dose-dependent increase in the percent of patients with a positive ALC slope, favoring the 10 mg/kg dose, and are consistent with the observation that more than 90% of patients treated with this dose had a Cminₛₛ higher than the defined target value for blockade of CTLA-4. Across studies 007, 008, and 022, patients with clinical benefit had a greater mean rate of ALC change (slope) than did patients without clinical benefit (P=0.0013). Importantly, in these three studies, no patient with a negative ALC slope over the induction dosing period had clinical benefit. These associations were confirmed in the independent study, CA184-004: patients with benefit had a greater mean slope (P=0.00042), and only 1 patient with a (slightly) negative ALC slope had clinical benefit. Baseline ALC was not associated with clinical benefit in any of the analyses.

### Conclusion

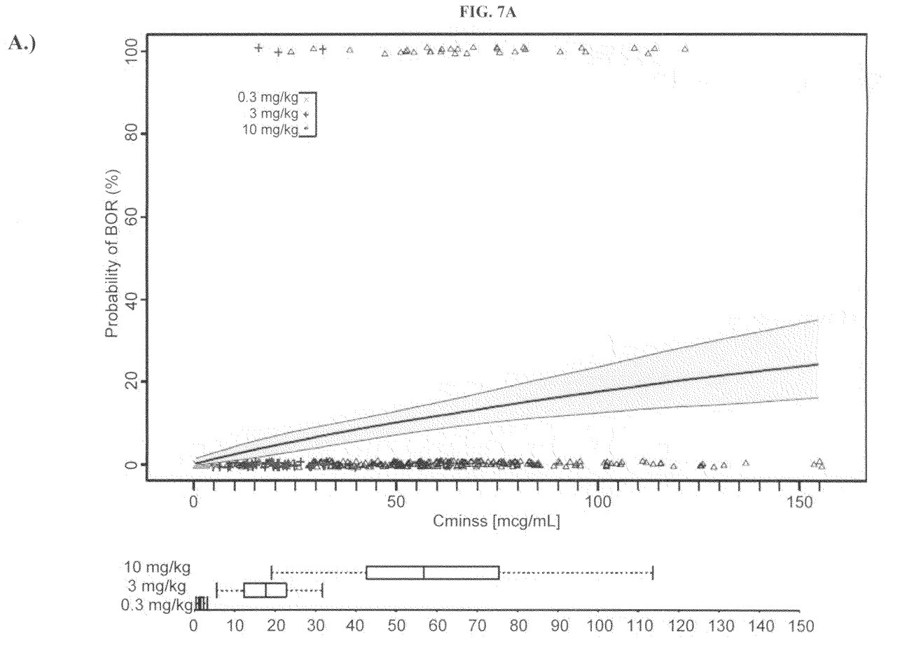

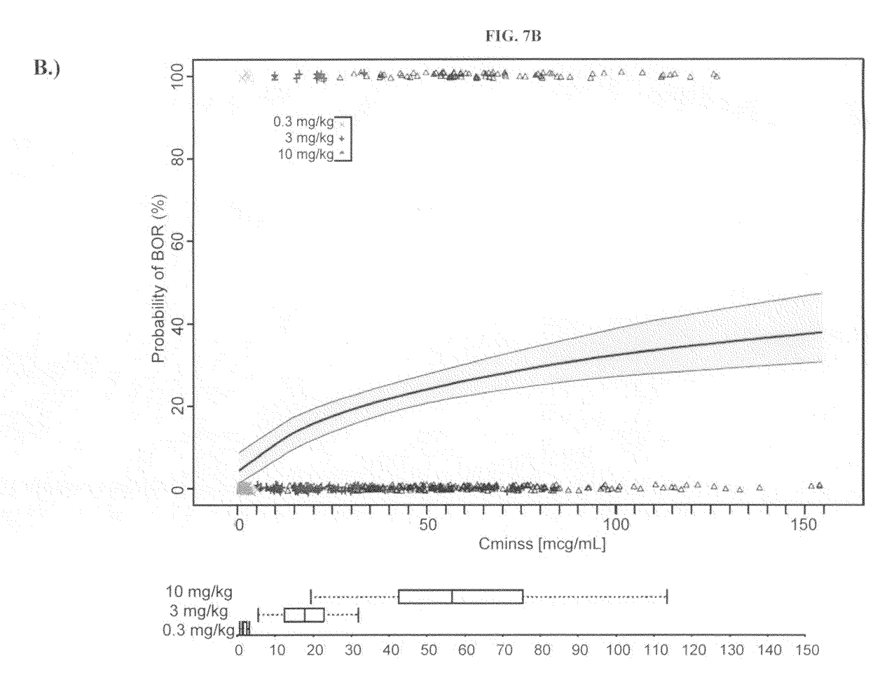

Ipilimumab has demonstrated antitumor effects in patients with advanced melanoma in phase II clinical trials (Hamid et al., J Clin Oncol 2008;26(19s):abstr 9025; O'Day et al., J Clin Oncol 2008;26(19s):abstr 9021). With endpoints of BOR rate and overall survival, the results of study CA184-022 provide evidence that an ipilimumab dose of 10 mg/kg offers the highest benefit-to-risk ratio (Hamid et al., J Clin Oncol 2008;26(19s):abstr 9025;). The population pharmacokinetics analysis presented in this report further confirm that, based on the target trough concentration, 10 mg/kg is an effective ipilimumab dose. Although the number of response events at the higher Cminₛₛ is low, the shape of the curves suggest that doses higher than 10 mg/kg may result in only incremental increases in the probability of BOR (see FIG. 7). Overall, these results support the selection of 10 mg/kg ipilimumab as the dose for phase III clinical trials.

Peripheral biomarkers of immune activation are easier to measure, yet it is unclear whether they are representative of the tumor microenvironment and can therefore be used to predict clinical benefit with ipilimumab or other immunotherapeutic agents. For example, high levels of peripheral tumor antigen-specific CD8⁺ T cells do not predict an antitumor response following cancer vaccination in patients with melanoma (Rosenberg et al., J Immunol 2005;175:6169-76). Inconsistent results have been reported as to whether higher ALC at baseline is predictive of benefit from anti-CD20 antibody (rituximab) therapy in non-Hodgkin's lymphomas (Behl et al., Br J Haematol 2007;137:409-15, Oki et al., Eur J Haematol 2008;81:448-53), but higher ALC has been observed in patients with advanced melanoma who showed antitumor responses following intralesional immunotherapy (Ridolfi et al., Hepatogastroenterology 2002;49, 335-39). How ipilimumab-induced changes in peripheral blood ALC relate to changes in the frequency of T cells in the tumor microenvironment are beyond the scope of the current studies.

Although questions have been raised as to whether peripheral biomarkers can be used to predict which patients will benefit from immunotherapy, our results provide evidence that changes in ALC are associated with ipilimumab clinical activity in melanoma. From a cross-study analysis of three multinational, phase II clinical trials in patients with advanced melanoma, the inventors have demonstrated: (i) an increase in probability of an antitumor response as ipilimumab exposure (e.g., Cmin) increases, and (ii) a positive association between rate of change in ALC and clinical benefit from ipilimumab at 10 mg/kg. Although pharmacokinetic parameters have not been evaluated from the fourth study, CA184-004, the association between ALC and clinical benefit was confirmed.

Our results further show that patients with a negative ALC slope are unlikely to experience a clinical benefit. Thus, a negative ALC slope could be used for negative enrichment, i.e., to identify those patients unlikely to benefit from continued ipilimumab therapy (and in which treatment could be terminated) or to identify those patients who may benefit from higher doses of ipilimumab or combinations of other therapies with ipilimumab. This result is consistent with another study demonstrating that low lymphocyte counts in patients with advanced cancer is a negative factor for survival (Viganó et al., Arch Intern Med 2000;160:861-68). Future studies will determine whether there is an association between changes in ALC and survival in ipilimumab-treated patients with advanced melanoma. In summary, ALC is a measurement derived from routine safety labs and could therefore be readily integrated into any treatment program with ipilimumab, and should be further explored as a predictive biomarker for immunotherapeutic agents.

TABLE 2

| | | | | | | Fraction |
|---|---|---|---|---|---|---|
| | | | | Mean | SD | Negative |
| Study | Dose | Group | N | Slope | Slope | Slope |
| 004 | 3.0 mg/kg | Benefit | 6 | 0.030 | 0.030 | 0.17 |
| | | Non-Benefit | 21 | −0.019 | 0.068 | 0.52 |
| | | Unknown | 5 | 0.028 | 0.026 | 0 |
| | 10.0 mg/kg | Benefit | 6 | 0.153 | 0.124 | 0 |
| | | Non-Benefit | 23 | 0.030 | 0.063 | 0.30 |
| | | Unknown | 4 | −0.036 | 0.172 | 0.50 |

### Example 3

### Methods of Measuring Absolute Lymphocyte Count in a Patient

A number of methods are known in the art for measuring absolute lymphocyte counts. One non-limiting example is provided. Briefly, patient blood samples are obtained and the total number of white blood cells are counted per microliter. The percentage of lymphocytes from the total number of white blood cells is determined (using hemocytometer, flow cytometry, or other methods known in the art), and multiplied by the total number of white blood cells to arrive at the “absolute” lymphocyte count.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

What is claimed is:

1. A method for predicting the likelihood a patient with cancer will have a favorable response to therapy with a co-stimulatory pathway modulator, comprising the steps of:
(i) measuring absolute lymphocyte count of patient samples collected over time subsequent, and optionally prior, to administration of said therapy; and
(ii) calculating a slope of said absolute lymphocyte count, wherein patients that have a negative slope have a lower likelihood of achieving a favorable response to said therapy.

2. The method of claim 1 wherein said cancer is a solid tumor.

3. The method of claim 2 wherein said cancer is selected from the group consisting of: melanoma, prostate cancer, lung cancer, non-small cell lung cancer, and small cell lung cancer.

4. The method of claim 1, 2, or 3 wherein the co-stimulatory pathway modulator is a CTLA-4 antagonist.

5. The method of claim 4 wherein the CTLA-4 antagonist is selected from the group consisting of: ipilimumab and tremelimumab.

6. A method of treating an individual suffering from cancer with a therapy with a co-stimulatory pathway modulator comprising the steps of:
(i) measuring absolute lymphocyte count of patient samples collected over time subsequent, and optionally prior, to administration of said therapy; and
(ii) calculating a slope of said absolute lymphocyte count, wherein patients that have a negative slope may require a more aggressive dosing regimen of a therapeutically acceptable amount of said therapy, either alone or in combination with other agents to treat said cancer.

7. The method of claim 6 wherein said cancer is a solid tumor.

8. The method of claim 7 wherein said cancer is selected from the group consisting of: melanoma, prostate cancer, lung cancer, non-small cell lung cancer, and small cell lung cancer.

9. The method of claim 6, 7, or 8 wherein the co-stimulatory pathway modulator is a CTLA-4 antagonist.

10. The method of claim 9 wherein the CTLA-4 antagonist is selected from the group consisting of: ipilimumab and tremelimumab.

11. The method of claim 10, wherein a recommended dose for said co-stimulatory pathway modulator is administered at a dosage of about 0.1 to 15 mg/kg once every three weeks, and wherein said more aggressive dosing regimen is administered at a dosage greater than the recommended dose or greater than about 10 mg/kg once every three weeks.

12. The method of claim 10, wherein said other agent is selected from the group consisting of: a tubulin stabilizing agent, a second co-stimulatory pathway modulator, a taxane, paclitaxel, an epothilone, IXEMPRA™, PROVENGE®, Bevacizumab, Dacarbazine, Paraplatin; Budesonide; an inhibitor of CD137; and steroids.

13. A kit for use in determining a treatment regimen for an individual with cancer, comprising:
(i) a means for measuring absolute lymphocyte counts over time, and
(ii) a means for calculating a slope for said absolute lymphocyte counts; and optionally instructions for use and interpretation of the kit results, wherein said treatment strategy comprises administration of a therapeutically effective amount of a co-stimulatory pathway modulator.

14. The kit of claim 13, wherein said treatment regimen comprises administration of a therapeutically effective amount of a therapy selected from the group consisting of: ipilimumab and tremelimumab.


SEQUENCE LISTING (4 sequences)

SEQ ID NO: 1 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 2 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 3 - protein, 9 aa, Homo sapiens
IMDQVPFSV

SEQ ID NO: 4 - protein, 9 aa, Homo sapiens
YLEPGPVTV